(19) United States
(12) Patent Application Publication
(10) Pub. No.: US 2020/0205682 A1
(43) Pub. Date: Jul. 2, 2020

(54) PULSE WAVE MEASUREMENT DEVICE, BLOOD PRESSURE MEASUREMENT DEVICE, EQUIPMENT, METHOD FOR MEASURING PULSE WAVE, AND METHOD FOR MEASURING BLOOD PRESSURE

(71) Applicants: Omron Corporation, Kyoto (JP); OMRON HEALTHCARE Co., Ltd., Kyoto (JP)

(72) Inventors: Hisashi Ozawa, Kyoto-shi (JP); Keisuke Saito, Osaka (JP); Keigo Kamada, Tokyo (JP); Yasuhiro Kawabata, Kyoto (JP)

(73) Assignees: Omron Corporation, Kyoto (JP); OMRON HEALTHCARE Co., Ltd., Kyoto (JP)

(21) Appl. No.: 16/813,280

(22) Filed: Mar. 9, 2020

Related U.S. Application Data
(63) Continuation of application No. PCT/JP2018/024045, filed on Jun. 25, 2018.

(30) Foreign Application Priority Data
Sep. 12, 2017 (JP) 2017-175089

(51) Int. Cl. A61B 5/024; A61B 5/00; A61B 5/021; A61B 5/05

(52) U.S. Cl. CPC A61B 5/02438; A61B 5/681; A61B 5/05; A61B 5/02444; A61B 5/6843; A61B 5/02125

(57) ABSTRACT

A pulse wave measurement device of the present invention includes a transmitter configured to emit a radio wave toward a measurement target site, a receiver configured to receive the radio wave reflected from the measurement target site, and a pulse wave detector configured to detect, based on an output of the receiver, a pulse wave signal representing a pulse wave of an artery passing through the measurement target site. The radio wave emitted from the transmitter has a bandwidth narrowed by a predetermined bandwidth index.

### CROSS-REFERENCE TO RELATED APPLICATION

This is a continuation application of International Application No. PCT/JP2018/024045, with an International filing date of Jun. 25, 2018, which claims priority of Japanese Patent Application No. 2017-175089 filed on Sep. 12, 2017, the entire content of which is hereby incorporated by reference.

### TECHNICAL FIELD

The present invention relates to a pulse wave measurement device, and more particularly, to a pulse wave measurement device that emits a radio wave toward a measurement target site of a living body or receives a radio wave from the measurement target site for measuring a pulse wave. The present invention also relates to a blood pressure measurement device including such a pulse wave measurement device. Furthermore, the present invention relates to an apparatus including such a blood pressure measurement device. Furthermore, the present invention relates to a pulse wave measurement method for measuring a pulse wave by such a pulse wave measurement device, and a blood pressure measurement method for measuring blood pressure by such a blood pressure measurement device.

### BACKGROUND ART

As this type of pulse wave measurement device, for example, as disclosed in Patent Document 1 (JP 5879407 B2 specification), there has been conventionally known a device including a transmitting (emitting) antenna and a receiving antenna that face to a measurement target site. The transmitting antenna emits a radio wave (measuring signal) toward a measurement target site (target object), and the receiving antenna receives the radio wave reflected from this measurement target site (reflected signal) for measuring a pulse wave. A square wave (pulse wave) has been used as the radio wave (measuring signal) that is aimed at a blood vessel.

### SUMMARY OF THE INVENTION

Meanwhile, the square wave (pulse wave), as is known, includes high-order wide frequency components. As a result, the reflected signal reflected from the measurement target site also includes wide frequency components. Accordingly, analyzing this reflected signal for detecting a change in blood vessel diameter means analyzing the wide frequency components included in the reflected signal. Consequently, there is a problem that complicated signal processing such as the Fourier transform has to be performed for achieving a sufficiently high S/N ratio.

Thus, an object of the present invention is to provide a pulse wave measurement device that can achieve a high S/N ratio without requiring complicated signal processing such as the Fourier transform. Another object of the present invention is to provide a blood pressure measurement device including such a pulse wave measurement device. Another object of the present invention is to provide an apparatus including such a blood pressure measurement device. Another object of the present invention is to provide a pulse wave measurement method for measuring a pulse wave by such a pulse wave measurement device, and a blood pressure measurement method for measuring blood pressure by such a blood pressure measurement device.

In the exemplary pulse wave measurement device of the present disclosure, a pulse wave measurement device configured to measure a pulse wave of a measurement target site of a living body, the pulse wave measurement device includes:

a transmitter configured to emit a radio wave toward the measurement target site;

a receiver configured to receive the radio wave reflected from the measurement target site; and

a pulse wave detector configured to detect, based on an output of the receiver, a pulse wave signal representing a pulse wave of an artery passing through the measurement target site and/or a tissue adjacent to the artery, wherein

the radio wave emitted from the transmitter has a bandwidth narrowed by a predetermined bandwidth index.

In the present specification, the “measurement target site” may be not only a rod-shaped portion such as an upper limb (wrist, upper arm, etc.) or a lower limb (ankle, etc.) but also a trunk.

The “tissue adjacent to an artery” refers to a portion of a living body that is adjacent to the artery and is periodically displaced under the influence of a pulse wave (that causes expansion and contraction of a blood vessel) of the artery.

The “bandwidth index” refers to, for example, an occupied bandwidth representing a range occupied by radio wave frequencies, a fractional bandwidth obtained by dividing the occupied bandwidth by a center frequency (f₀) (=occupied bandwidth/center frequency (f₀)), or the like. The bandwidth index is not limited to these, and another type of bandwidth index is possible.

When the “fractional bandwidth” is used as the “bandwidth index”, the fractional bandwidth is preferably 0.03 or smaller.

In another aspect, the exemplary blood pressure measurement device of the present disclosure configured to measure blood pressure of a measurement target site of a living body, comprises:

two sets of the pulse wave measurement devices,

a belt of the two sets is integrally formed,

the transmitter and the receiver of a first set out of the two sets are disposed separately from the transmitter and the receiver of a second set in a width direction of the belt,

in a wearing state where the belt is worn around an outer surface of the measurement target site, the transmitter and the receiver of the first set meet an upstream portion of an artery passing through the measurement target site, while the transmitter and the receiver of the second set meet a downstream portion of the artery,

in each of the two sets, the transmitter emits a radio wave toward the measurement target site and the receiver receives the radio wave reflected from the measurement target site,

in each of the two sets, the pulse wave detector acquires, based on an output of the receiver, a pulse wave signal representing a pulse wave of the artery passing through the measurement target site and/or a tissue adjacent to the artery, and

the blood pressure measurement device comprises:

a time difference acquisition unit configured to acquire a time difference between the pulse wave signals acquired by the pulse wave detectors of the two sets as a pulse transit time; and

a first blood pressure calculator configured to calculate a blood pressure value based on the pulse transit time acquired by the time difference acquisition unit using a predetermined correspondence equation between pulse transit time and blood pressure.

In another aspect, the exemplary apparatus of the present disclosure comprises the pulse wave measurement device, or the blood pressure measurement device.

In another aspect, the exemplary pulse wave measurement method of the present disclosure for measuring a pulse wave of a measurement target site of a living body using the pulse wave measurement device comprises:

wearing the belt around an outer surface of the measurement target site to make the transmitter and the receiver meet an artery passing through the measurement target site;

emitting, by the transmitter, a radio wave having a bandwidth narrowed by a predetermined bandwidth index toward the measurement target site, and receiving, by the receiver, the radio wave reflected from the measurement target site; and

detecting, by the pulse wave detector, based on an output of the receiver, a pulse wave signal representing a pulse wave of the artery passing through the measurement target site and/or a tissue adjacent to the artery.

In another aspect, the exemplary blood pressure measurement method of the present disclosure for measuring blood pressure of a measurement target site of a living body using the blood pressure measurement device comprises:

wearing the belt around an outer surface of the measurement target site to make the transmitter and the receiver of the first set out of the two sets meet an upstream portion of an artery passing through the measurement target site, and equally to make the transmitter and the receiver of the second set meet a downstream portion of the artery;

in each of the two sets, emitting, by the transmitter, a radio wave having a bandwidth narrowed by a predetermined bandwidth index toward the measurement target site, and receiving, by the receiver, the radio wave reflected from the measurement target site;

in each of the two sets, acquiring, by the pulse wave detector, based on an output of the receiver, a pulse wave signal representing a pulse wave of the artery passing through the measurement target site and/or a tissue adjacent to the artery;

acquiring, by the time difference acquisition unit, a time difference between the pulse wave signals acquired by the pulse wave detectors of the two sets as a pulse transit time; and

calculating, by the first blood pressure calculator, a blood pressure value based on the pulse transit time acquired by the time difference acquisition unit using a predetermined correspondence equation between pulse transit time and blood pressure.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a perspective view showing an appearance of a wrist-type sphygmomanometer of an embodiment according to a pulse wave measurement device and a blood pressure measurement device of the present invention.

FIG. 2 is a diagram schematically showing, in a state where the sphygmomanometer is worn on a left wrist, a cross-section perpendicular to the longitudinal direction of the wrist.

FIG. 3 is a diagram showing, in the state where the sphygmomanometer is worn on the left wrist, a planar layout of a transmitting/receiving antenna group constituting first and second pulse wave sensors.

FIG. 4 is a diagram showing an overall block configuration of a control system of the sphygmomanometer.

FIG. 5 is a diagram showing a partial functional block configuration of the control system of the sphygmomanometer.

FIG. 6A is a diagram schematically showing, in the state where the sphygmomanometer is worn on the left wrist, a cross-section along the longitudinal direction of the wrist. FIG. 6B is a diagram showing waveforms of first and second pulse wave signals output by the first and second pulse wave sensors, respectively.

FIG. 7A is a diagram showing a block configuration implemented by a program for performing an oscillometric method in the sphygmomanometer.

FIG. 7B is a diagram showing an operation flow when the sphygmomanometer performs blood pressure measurement by the oscillometric method.

FIG. 8 is a diagram showing changes in cuff pressure and a pulse wave signal according to the operation flow in FIG. 9.

FIG. 9 is a diagram showing an operation flow according to a pulse wave measurement method and a blood pressure measurement method of an embodiment of the present invention, in which the sphygmomanometer performs pulse wave measurement to acquire a pulse transit time (PTT) and performs blood pressure measurement (estimation) based on the pulse transit time.

FIG. 10A is an operation flowchart of emitting a radio wave having a narrowed bandwidth to a measurement target site and receiving the radio wave from the measurement target site. FIG. 10B is an operation flowchart of shifting or sweeping a center frequency (f₀). FIG. 10C is an operation flowchart of intermittent transmission.

FIG. 11A is a diagram showing a waveform of a sine wave with a frequency of 24.050 GHz. FIG. 11B is a frequency spectrum diagram related to the sine wave (frequency of 24.050 GHz).

FIG. 12A is a diagram showing a waveform of a sine wave with a frequency of 24.250 GHz. FIG. 12B is a frequency spectrum diagram related to the sine wave (frequency of 24.250 GHz).

FIG. 13A is a diagram showing a waveform of an intermittent sine wave with a sine wave frequency of 24.250 GHz. FIG. 13B is a frequency spectrum diagram related to the intermittent sine wave.

FIG. 14A is a diagram showing a waveform of a continuous modulated wave with a carrier wave frequency of 24.050 GHz. FIG. 14B is a frequency spectrum diagram related to the continuous modulated wave.

FIG. 15A is a diagram showing a waveform of a frequency-shifted modulated wave with a carrier wave frequency of 24.250 GHz. FIG. 15B is a frequency spectrum diagram related to the frequency-shifted modulated wave.

FIG. 16A is a diagram showing a waveform of an intermittent modulated wave with a carrier wave frequency of 24.150 GHz. FIG. 16B is a frequency spectrum diagram related to the intermittent modulated wave.

FIG. 17A is a diagram showing a waveform of a pulse wave. FIG. 17B is a frequency spectrum diagram related to the pulse wave.

FIG. 18A is a partial enlarged view of the intermittent sine wave in FIG. 13A. FIG. 18B is a partial enlarged view of the continuous modulated wave in FIG. 14A.

FIG. 19A is a diagram showing a block configuration according to an embodiment in which a frequency is switched and shifted according to an operation flow in FIG. 20.

FIG. 19B is a diagram showing a block configuration according to an embodiment in which a frequency is shifted or swept based on a cross-correlation coefficient between a waveform of a pulse wave signal and a reference waveform according to an operation flow in FIG. 21.

FIG. 19C is a diagram showing a block configuration according to an embodiment in which a frequency is shifted or swept based on a cross-correlation coefficient between an output waveform of a first pulse wave signal and an output waveform of a second pulse wave signal according to an operation flow in FIG. 22.

FIG. 20 is the operation flowchart of shifting the frequency by switching the frequency based on a signal-to-noise ratio of a pulse wave signal.

FIG. 21 is the operation flowchart of shifting or sweeping the frequency based on the cross-correlation coefficient between the waveform of the pulse wave signal and the reference waveform.

FIG. 22 is the operation flowchart of shifting or sweeping the frequency based on the cross-correlation coefficient between the output waveform of the first pulse wave signal and the output waveform of the second pulse wave signal.

FIG. 23 is a diagram illustrating an equation representing a cross-correlation coefficient r between a data string {xi} and a data string {yi}.

### DESCRIPTION OF EMBODIMENTS

Hereinafter, exemplary embodiments of the present disclosure will be described in detail with reference to the drawings.

(Configuration of Sphygmomanometer)

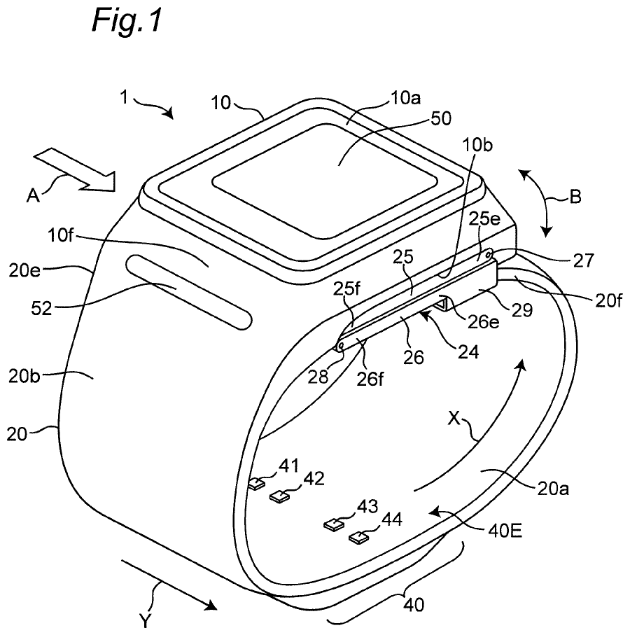

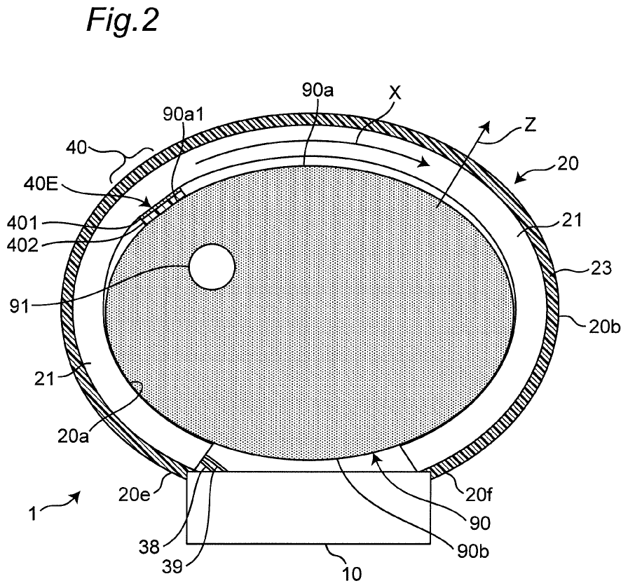

FIG. 1 shows a perspective view of an appearance of a wrist-type sphygmomanometer (generally indicated by reference sign 1) of an embodiment according to an exemplary pulse wave measurement device and an exemplary blood pressure measurement device of the present disclosure. FIG. 2 schematically shows a cross-section perpendicular to the longitudinal direction of a left wrist 90 in a state where the sphygmomanometer 1 is worn on the left wrist 90 as a measurement target site (hereinafter referred to as “wearing state”).

As shown in these figures, the sphygmomanometer 1 mainly includes a belt 20 that is worn around the user's left wrist 90 and a main body 10 that is integrally attached to the belt 20. Overall, the sphygmomanometer 1 is configured as a counterpart of the blood pressure measurement device including two sets of the pulse wave measurement devices.

As can be seen from FIG. 1, the belt 20 has an elongated band-like shape to be wound around the left wrist 90 along the circumferential direction. The belt 20 includes an inner peripheral surface 20a that contacts the left wrist 90, and an outer peripheral surface 20b opposite to the inner peripheral surface 20a. The dimension in a width direction Y (width dimension) of the belt 20 is set to about 30 mm in this example.

The main body 10 is integrally installed at one end portion 20e of the belt 20 in the circumferential direction by integral molding in this example. The belt 20 and the main body 10 may be formed separately. Then, the main body 10 may be integrally attached to the belt 20 via an engaging member (for example, a hinge or the like). In this example, a portion where the main body 10 is disposed is scheduled to meet a back-side surface (a surface on the back side of a hand) 90b of the left wrist 90 in the wearing state (see FIG. 2). FIG. 2 shows a radial artery 91 in the left wrist 90 passing through the vicinity of a palm-side surface (a surface on the palm side of a hand) 90a as an outer surface.

As can be seen from FIG. 1, the main body 10 has a three-dimensional shape having a thickness in a direction perpendicular to the outer peripheral surface 20b of the belt 20. The main body 10 is formed to be compact and thin so as not to disturb user's daily activities. In this example, the main body 10 has a truncated-quadrangular-pyramid-shaped outline projecting outward from the belt 20.

A display unit 50 that serves as a display screen is installed on a top surface (a surface on the side farthest from the measurement target site) 10a of the main body 10. Further, an operation unit 52 for inputting an instruction from the user is installed along a side surface (a side surface on the left front side in FIG. 1) 10f of the main body 10.

A transmitter/receiver 40 constituting first and second pulse wave sensors is fitted on a portion between the one end portion 20e and the other end portion 20f of the belt 20 in the circumferential direction. On the inner peripheral surface 20a of the belt 20 at the portion where the transmitter/receiver 40 is disposed, four transmitting/receiving antennas 41 to 44 (generally referred to as “transmitting/receiving antenna group” and represented by reference sign 40E) are mounted separately from each other in the width direction Y of the belt 20 (which will be described in detail later). In this example, the portion where the transmitting/receiving antenna group 40E is disposed in a longitudinal direction X of the belt 20 is scheduled to meet the radial artery 91 of the left wrist 90 in the wearing state (see FIG. 2).

As shown in FIG. 1, a bottom surface (a surface on the side closest to the measurement target site) 10b of the main body 10 is connected to the end portion 20f of the belt 20 by a threefold buckle 24. The buckle 24 includes a first plate-like member 25 disposed on the outer peripheral side and a second plate-like member 26 disposed on the inner peripheral side. One end portion 25e of the first plate-like member 25 is rotatably attached to the main body 10 via a connecting rod 27 extending along the width direction Y. The other end portion 25f of the first plate-like member 25 is rotatably attached to one end portion 26e of the second plate-like member 26 via a connecting rod 28 extending along the width direction Y. The other end portion 26f of the second plate-like member 26 is fixed in the vicinity of the end portion 20f of the belt 20 by a fixing portion 29. Note that the attachment position of the fixing portion 29 in the longitudinal direction X of the belt 20 (corresponding to the circumferential direction of the left wrist 90 in the wearing state) is variably set in advance to fit the circumferential length of the user's left wrist 90. Thus, the sphygmomanometer 1 (belt 20) is configured in a substantially annular shape as a whole, and the bottom surface 10b of the main body 10 and the end portion 20f of the belt 20 can be opened and closed in a direction of an arrow B using the buckle 24.

When wearing the sphygmomanometer 1 on the left wrist 90, the user puts the left hand through the belt 20 in a direction indicated by an arrow A in FIG. 1 with the buckle 24 open and the diameter of the annular belt 20 larger. Then, as shown in FIG. 2, the user adjusts an angular position of the belt 20 around the left wrist 90 to position the transmitter/receiver 40 of the belt 20 on the radial artery 91 passing through the left wrist 90. As a result, the transmitting/receiving antenna group 40E of the transmitter/receiver 40 is in a contact with a portion 90a1 of the palm-side surface 90a of the left wrist 90 corresponding to the radial artery 91. In this state, the user closes the buckle 24 for fixing. Thus, the user wears the sphygmomanometer 1 (belt 20) on the left wrist 90.

As shown in FIG. 2, in this example, the belt 20 includes a band-like body 23 forming the outer peripheral surface 20b, and a press cuff 21 as a pressing member attached along the inner peripheral surface of the band-like body 23. The band-like body 23 is made of a plastic material (in this example, silicone resin). In this example, the band-like body 23 is flexible in a thickness direction Z and hardly stretchable (substantially non-stretchable) in the longitudinal direction X (corresponding to the circumferential direction of the left wrist 90). In this example, the press cuff 21 is formed as a fluid bag by making two stretchable polyurethane sheets face each other in the thickness direction Z and welding the peripheral portions of the sheets. On the inner peripheral surface 20a of the press cuff 21 (belt 20), the transmitting/receiving antenna group 40E of the transmitter/receiver 40 is disposed at the portion that meets the radial artery 91 of the left wrist 90 as described above.

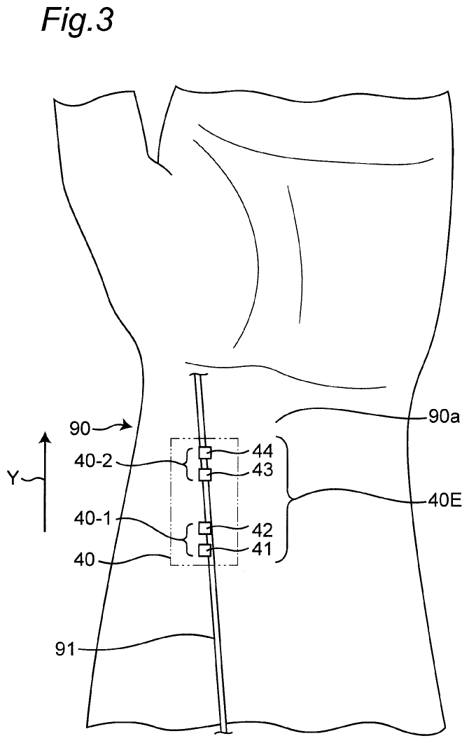

In this example, as shown in FIG. 3, in the wearing state, the transmitting/receiving antenna group 40E of the transmitter/receiver 40 is in a state where the transmitting/receiving antennas are arranged separately from each other approximately along the longitudinal direction of the left wrist 90 (corresponding to the width direction Y of the belt 20) to meet the radial artery 91 of the left wrist 90. In this example, the transmitting/receiving antenna group 40E includes the transmitting antennas 41, 44 disposed on both sides in the width direction Y in a range occupied by the transmitting/receiving antenna group 40E, and the receiving antennas 42, 43 disposed between the transmitting antennas 41, 44. The transmitting antenna 41 and the receiving antenna 42 that receives a radio wave from the transmitting antenna 41 constitute a pair of transmitting/receiving antennas (41, 42) (the pair is shown in parentheses; the same hereinafter) of a first set. The transmitting antenna 44 and the receiving antenna 43 that receives a radio wave from the transmitting antenna 44 constitute a pair of transmitting/receiving antennas (44, 43) of a second set. In this arrangement, the transmitting antenna 41 is closer to the receiving antenna 42 than the transmitting antenna 44. Further, the transmitting antenna 44 is closer to the receiving antenna 43 than the transmitting antenna 41. Accordingly, interference between the pair of transmitting/receiving antennas (41, 42) of the first set and the pair of transmitting/receiving antennas (44, 43) of the second set can be reduced. The antenna arrangement order is not limited to the order of transmitting antenna, receiving antenna, receiving antenna, and transmitting antenna as in this example, and may be an order of receiving antenna, transmitting antenna, transmitting antenna, and receiving antenna.

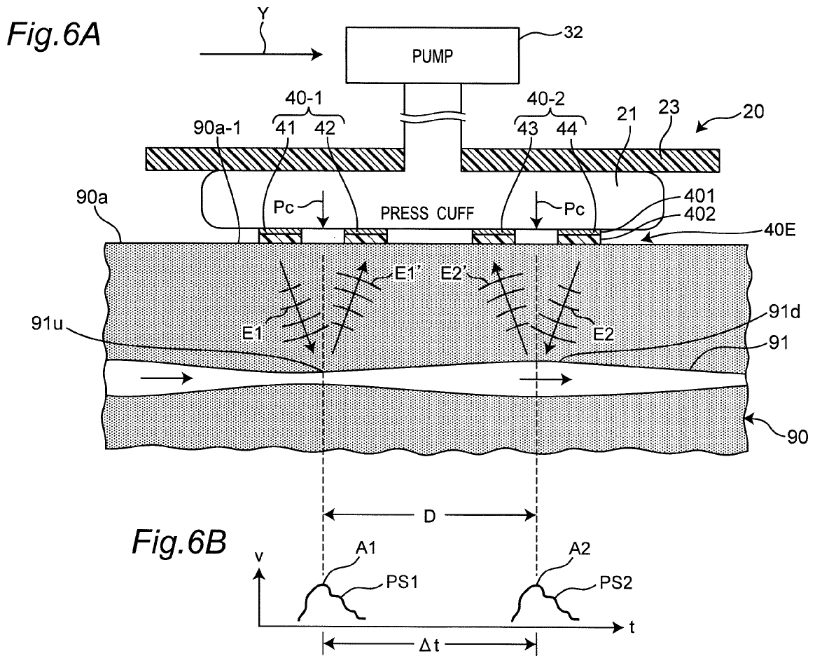

In this example, one transmitting or receiving antenna has, in a surface direction (meaning a direction along the outer peripheral surface of the left wrist 90 in FIG. 3), a square shape with a side of 3 mm in both vertical and horizontal directions (this shape in the surface direction is referred to as “pattern shape”) to emit or receive a radio wave having a frequency of 24 GHz band. In this example, a distance between centers of the transmitting antenna 41 and the receiving antenna 42 of the first set in the width direction Y of the belt 20 is set within a range of 5 mm to 10 mm. Similarly, in this example, a distance between centers of the transmitting antenna 44 and the receiving antenna 43 of the second set in the width direction Y of the belt 20 is set within a range of 5 mm to 10 mm. Further, a distance D (see FIG. 6) between midpoint of the pair of transmitting/receiving antennas (41, 42) of the first set and midpoint of the pair of transmitting/receiving antennas (44, 43) of the second set in the width direction Y of the belt 20 is set to 20 mm in this example. The distance D corresponds to a substantial interval between the pair of transmitting/receiving antennas (41, 42) of the first set and the pair of transmitting/receiving antennas (44, 43) of the second set. Note that the length of the distance D or the like is an example, and an optimal length may be appropriately selected depending on the size of the sphygmomanometer and the like.

Further, as shown in FIG. 2, in this example, the transmitting/receiving antenna group 40E includes a conductor layer 401 attached to the belt 20 for emitting or receiving a radio wave and a dielectric layer 402 attached along a surface of the conductor layer 401 on a side that faces the left wrist 90. The conductor layer 401 and the dielectric layer 402 are sequentially laminated in the thickness direction Z (each of the transmitting and receiving antennas has the same configuration). In this example, the pattern shape of the dielectric layer 402 is set to be the same as the pattern shape of the conductor layer 401, but may be different. In the wearing state where the transmitting/receiving antenna group 40E is worn on the left wrist 90, the dielectric layer 402 works as a spacer to keep a distance (a distance in the thickness direction Z) between the palm-side surface 90a of the left wrist 90 and the conductor layer 401 constant.

In this example, the conductor layer 401 is made of metal (for example, copper or the like). In this example, the dielectric layer 402 is made of polycarbonate.

Such a transmitting/receiving antenna group 40E can be formed to be flat along the outer peripheral surface of the left wrist 90. Therefore, in the sphygmomanometer 1, the belt 20 can be formed to be thin as a whole. In this example, the thickness of the conductor layer 401 is set to 30 μm, and the thickness of the dielectric layer 402 is set to 2 mm.

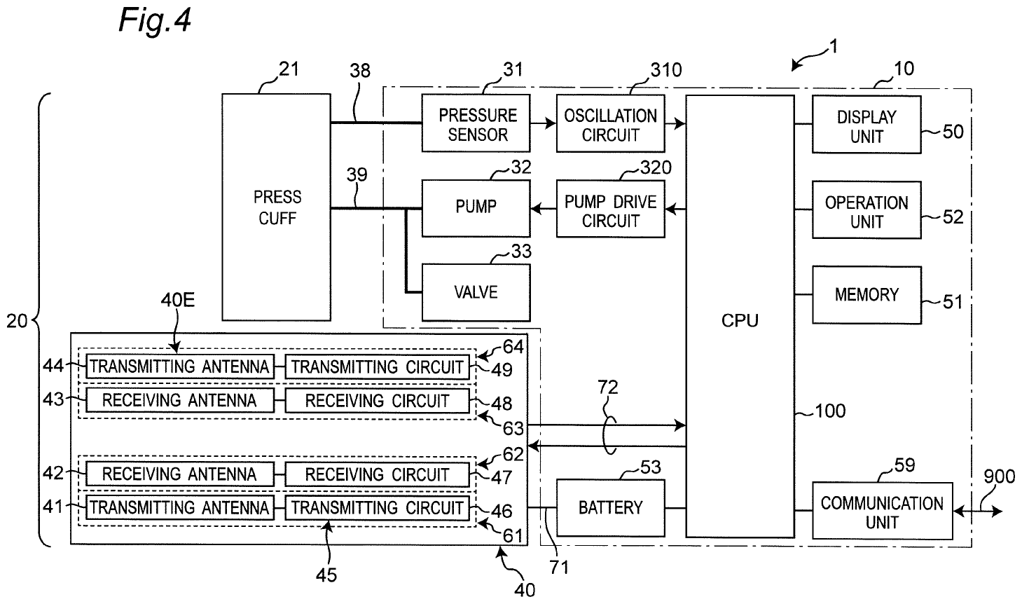

FIG. 4 shows an overall block configuration of a control system of the sphygmomanometer 1. In the main body 10 of the sphygmomanometer 1, there are mounted, in addition to the display unit 50 and the operation unit 52 described above, a central processing unit (CPU) 100 as a controller, a memory 51 as a storage unit, a communication unit 59, a pressure sensor 31, a pump 32, a valve 33, an oscillation circuit 310 that converts an output from the pressure sensor 31 into frequency, and a pump drive circuit 320 that drives the pump 32. Further, in the transmitter/receiver 40, there is mounted, in addition to the transmitting/receiving antenna group 40E described above, a transmitting/receiving circuit group 45 controlled by the CPU 100.

In this example, the display unit 50 is constituted by an organic electro luminescence (EL) display, and displays information on blood pressure measurement such as a blood pressure measurement result or other information in response to a control signal from the CPU 100. The display unit 50 is not limited to an organic EL display, and may be another type of display unit such as a liquid cristal display (LCD), for example.

In this example, the operation unit 52 is constituted by a push-type switch, and inputs an operation signal in response to a user's instruction to start or stop the blood pressure measurement to the CPU 100. Note that the operation unit 52 is not limited to a push-type switch, and may be a pressure-sensitive (resistive) or proximity (capacitive) touch-panel-type switch, for example. Further, a microphone (not shown) may be included for inputting an instruction to start the blood pressure measurement by user's voice.

The memory 51 non-temporarily stores data of a program for controlling the sphygmomanometer 1, data used for controlling the sphygmomanometer 1, setting data for setting various functions of the sphygmomanometer 1, data of measurement results of blood pressure values, and the like. The memory 51 is also used as a work memory when the program is executed or the like.

The CPU 100 performs various functions as the controller in accordance with the program for controlling the sphygmomanometer 1 stored in the memory 51. For example, when executing blood pressure measurement by the oscillometric method, the CPU 100 performs, in response to an instruction to start the blood pressure measurement from the operation unit 52, control of driving the pump 32 (and the valve 33) based on a signal from the pressure sensor 31. In this example, the CPU 100 also performs control of calculating a blood pressure value based on the signal from the pressure sensor 31.

The communication unit 59 is controlled by the CPU 100 to transmit predetermined information to an external device via a network 900, and receive information from the external device via the network 900 to deliver the information to the CPU 100. The communication via the network 900 may be wireless or wired. In this embodiment, the network 900 is the Internet. However, the network 900 is not limited to this and may be another type of network such as an in-hospital local area network (LAN) or one-to-one communication using a USB cable or the like. The communication unit 59 may include a micro USB connector.

The pump 32 and the valve 33 are connected to the press cuff 21 via an air pipe 39. The pressure sensor 31 is connected to the press cuff 21 via an air pipe 38. The air pipes 39, 38 may be a single common pipe. The pressure sensor 31 detects pressure within the press cuff 21 via the air pipe 38. In this example, the pump 32 is constituted by a piezoelectric pump, and supplies air as a pressurizing fluid into the press cuff 21 through the air pipe 39 to increase the pressure (cuff pressure) within the press cuff 21. The valve 33 is mounted on the pump 32 and is controlled to open and close depending on an on/off action of the pump 32. That is, the valve 33 closes to enclose the air in the press cuff 21 when the pump 32 is turned on, while the valve 33 opens to discharge the air in the press cuff 21 to the atmosphere through the air pipe 39 when the pump 32 is turned off. The valve 33 has a check valve function, and the discharged air does not flow backward. The pump drive circuit 320 drives the pump 32 based on a control signal from the CPU 100.

In this example, the pressure sensor 31 is a piezoresistive pressure sensor. The pressure sensor 31 detects the pressure of the belt 20 (press cuff 21), which is a pressure based on the atmospheric pressure (zero) in this example, through the air pipe 38 to output the detected pressure as a time series signal. The oscillation circuit 310 oscillates based on an electric signal value based on an electric resistance change due to the piezoresistance effect of the pressure sensor 31 to output a frequency signal having a frequency depending on the electric signal value of the pressure sensor 31 to the CPU 100. In this example, the output of the pressure sensor 31 is used for controlling the pressure of the press cuff 21 and calculating a blood pressure value (including systolic blood pressure (SBP) and diastolic blood pressure (DBP)) by the oscillometric method.

A battery 53 supplies power to each of the parts mounted on the main body 10 including, in this example, the CPU 100, the pressure sensor 31, the pump 32, the valve 33, the display unit 50, the memory 51, the communication unit 59, the oscillation circuit 310, and the pump drive circuit 320. The battery 53 also supplies power to the transmitting/receiving circuit group 45 of the transmitter/receiver 40 through a wiring 71. The wiring 71 is extended between the main body 10 and the transmitter/receiver 40 along the longitudinal direction X of the belt 20 in a state where the wiring 71, together with a signal wiring 72, is interposed between the band-like body 23 and the press cuff 21 of the belt 20.

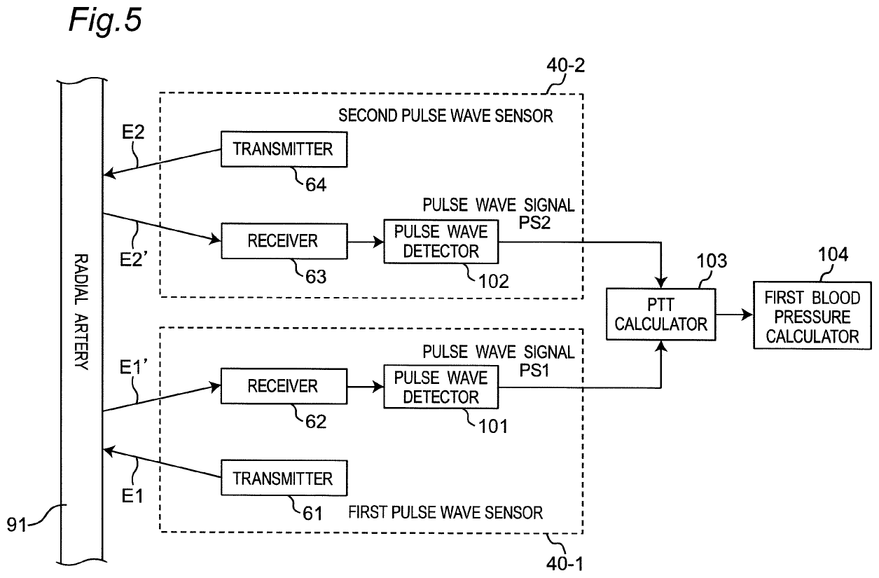

The transmitting/receiving circuit group 45 of the transmitter/receiver 40 includes transmitting circuits 46, 49 respectively connected to the transmitting antennas 41, 44, and receiving circuits 47, 48 respectively connected to the receiving antennas 42, 43. Here, the transmitting antenna 41 and the transmitting circuit 46 constitute a transmitter 61. The transmitting antenna 44 and the transmitting circuit 49 constitute a transmitter 64. The receiving antenna 42 and the receiving circuit 47 constitute a receiver 62. The receiving antenna 43 and the receiving circuit 48 constitute a receiver 63. As shown in FIG. 5, the transmitters 61, 64 respectively emit radio waves E1, E2 having a frequency of 24 GHz band in this example via the transmitting antennas 41, 44 during operation. The receivers 62, 63 respectively receive the radio waves E1′, E2′ reflected from the left wrist 90 (more precisely, the portion corresponding to the radial artery 91 and/or the tissue adjacent to the radial artery 91) as the measurement target site via the receiving antennas 42, 43 to detect and amplify the received radio waves. Hereinafter, for simplicity, it is assumed that the reflected radio waves E1′, E2′ are radio waves reflected from the radial artery 91.

As will be described in detail later, pulse wave detectors 101, 102 shown in FIG. 5 respectively acquire, based on outputs of the receivers 62, 63, pulse wave signals PS1, PS2 representing pulse waves of the radial artery 91 passing through the left wrist 90. Further, a PTT calculator 103 as a time difference acquisition unit acquires a time difference between the pulse wave signals PS1, PS2 respectively acquired by two sets of the pulse wave detectors 101, 102 as a pulse transit time (PTT). A first blood pressure calculator 104 calculates a blood pressure value based on the pulse transit time acquired by the PTT calculator 103 using a predetermined correspondence equation between pulse transit time and blood pressure. Here, the CPU 100 realizes the pulse wave detectors 101, 102, the PTT calculator 103, and the first blood pressure calculator 104 by executing a predetermined program. The transmitter 61, the receiver 62, and the pulse wave detector 101 constitute a first pulse wave sensor 40-1 as the first set of pulse wave measurement device. The transmitter 64, the receiver 63, and the pulse wave detector 102 constitute a second pulse wave sensor 40-2 as the second set of pulse wave measurement device.

In the wearing state, as shown in FIG. 6A, the pair of transmitting/receiving antennas (41, 42) of the first set meets an upstream portion 91u of the radial artery 91 passing through the left wrist 90 in the longitudinal direction of the left wrist 90 (corresponding to the width direction Y of the belt 20). Meanwhile, the pair of transmitting/receiving antennas (44, 43) of the second set meets a downstream portion 91d of the radial artery 91. A signal acquired by the pair of transmitting/receiving antennas (41, 42) of the first set indicates a distance change between the upstream portion 91u of the radial artery 91 and the pair of transmitting/receiving antennas (41, 42) of the first set resulting from the pulse wave (that causes expansion and contraction of a blood vessel). A signal acquired by the pair of transmitting/receiving antennas (44, 43) of the second set indicates a distance change between the downstream portion 91d of the radial artery 91 and the pair of transmitting/receiving antennas (44, 43) of the second set resulting from the pulse wave. The pulse wave detector 101 of the first pulse wave sensor 40-1 and the pulse wave detector 102 of the second pulse wave sensor 40-2 respectively output the first pulse wave signal PS1 and the second pulse wave signal PS2 having a mountain-shaped waveform as shown in FIG. 6B in a time series manner based on outputs from the receiving circuits 47, 48.

In this example, reception levels of the receiving antennas 42, 43 are about 1 μW (−30 dBm in decibel value with respect to 1 mW). Output levels of the receiving circuits 47, 48 are about 1 volt. Further, respective peaks A1, A2 of the first pulse wave signal PS1 and the second pulse wave signal PS2 are about 100 mV to 1 volt.

Assume that pulse wave velocity (PWV) of blood flow in the radial artery 91 is in a range of 1000 cm/s to 2000 cm/s. Since the substantial interval D between the first pulse wave sensor 40-1 and the second pulse wave sensor 40-2 is set to D=20 mm, the time difference Δt between the first pulse wave signal PS1 and the second pulse wave signal PS2 is in a range of 1.0 ms to 2.0 ms.

In the above example, the case where there are two sets of pairs of transmitting/receiving antennas has been described. However, there may be three or more sets of pairs of transmitting/receiving antennas.

(Configuration and Operation of Blood Pressure Measurement by Oscillometric Method)

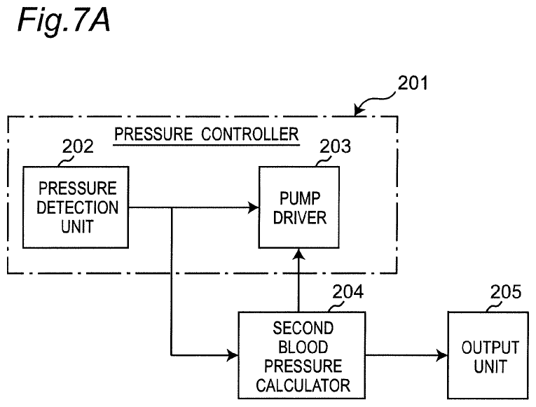

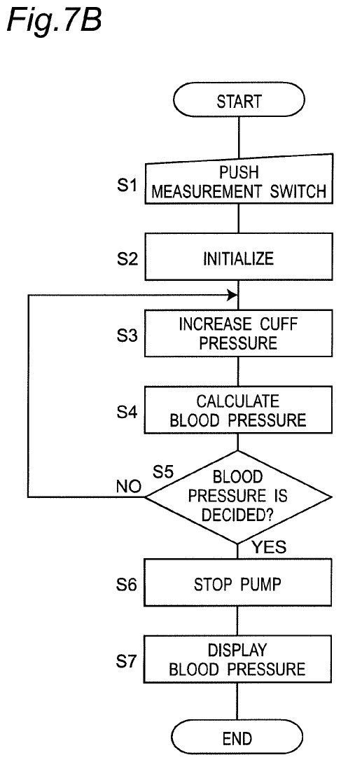

FIG. 7A shows a block configuration implemented by a program for performing the oscillometric method in the sphygmomanometer 1.

In this block configuration, a pressure controller 201, a second blood pressure calculator 204, and an output unit 205 are mainly implemented.

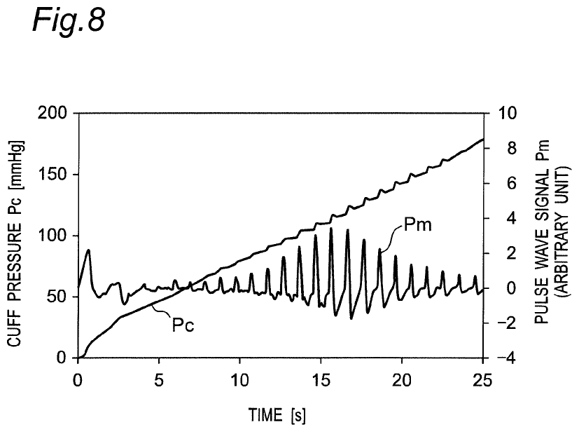

The pressure controller 201 further includes a pressure detection unit 202 and a pump driver 203. The pressure detection unit 202 processes the frequency signal input from the pressure sensor 31 through the oscillation circuit 310 to perform processing for detecting the pressure (cuff pressure) within the press cuff 21. The pump driver 203 performs processing for driving the pump 32 and the valve 33 through the pump drive circuit 320 based on the detected cuff pressure Pc (see FIG. 8). In this way, the pressure controller 201 controls the pressure by supplying air into the press cuff 21 at a predetermined pressurization rate.

The second blood pressure calculator 204 acquires a variation component of an arterial volume included in the cuff pressure Pc as the pulse wave signal Pm (see FIG. 8), and performs processing for calculating a blood pressure value (systolic blood pressure SBP and diastolic blood pressure DBP) based on the acquired pulse wave signal Pm by applying a publicly-known algorithm by the oscillometric method. When having completed the calculation of the blood pressure value, the second blood pressure calculator 204 causes the pump driver 203 to stop the processing.

The output unit 205 performs processing for displaying the calculated blood pressure value (systolic blood pressure SBP and diastolic blood pressure DBP) on the display unit 50 in this example.

FIG. 7B shows an operation flow (flow of the blood pressure measurement method) when the sphygmomanometer 1 performs the blood pressure measurement by the oscillometric method. The belt 20 of the sphygmomanometer 1 is assumed to be worn around the left wrist 90 in advance.

When the user instructs the blood pressure measurement by the oscillometric method using the push-type switch as the operation unit 52 installed on the main body 10 (step S1), the CPU 100 starts operation and initializes a memory area for processing (step S2). The CPU 100 further turns off the pump 32 via the pump drive circuit 320, opens the valve 33, and discharges the air in the press cuff 21. Subsequently, the CPU 100 performs control of setting a current output value of the pressure sensor 31 as a value corresponding to the atmospheric pressure (0 mmHg adjustment).

Subsequently, the CPU 100 works as the pump driver 203 of the pressure controller 201. The CPU 100 closes the valve 33 and then drives the pump 32 via the pump drive circuit 320 to perform control of feeding air into the press cuff 21. As a result, the press cuff 21 is inflated and the cuff pressure Pc (see FIG. 8) is gradually increased, which causes compression of the left wrist 90 as the measurement target site (step S3 in FIG. 7B).

In this pressurization process, the CPU 100 works as the pressure detection unit 202 of the pressure controller 201 for calculating a blood pressure value. The CPU 100 monitors the cuff pressure Pc using the pressure sensor 31 and acquires the variation component of the arterial volume generated in the radial artery 91 of the left wrist 90 as the pulse wave signal Pm as shown in FIG. 8.

Next, in step S4 in FIG. 7B, the CPU 100 works as the second blood pressure calculator. The CPU 100 attempts to calculate the blood pressure value (systolic blood pressure SBP and diastolic blood pressure DBP) based on the pulse wave signal Pm having been acquired at this point by applying a publicly-known algorithm by the oscillometric method.

When no blood pressure value can yet be calculated at this point due to lack of data (NO in step S5), the processing of steps S3 to S5 is repeated until the cuff pressure Pc reaches an upper limit pressure (for example, predetermined at 300 mmHg for safety).

When the blood pressure value can be calculated in this manner (YES in step S5), the CPU 100 performs control of stopping the pump 32 and opening the valve 33 to discharge the air in the press cuff 21 (step S6). Finally, the CPU 100 works as the output unit 205 to display a measurement result of the blood pressure value on the display unit 50 and record the measurement result in the memory 51 (step S7).

The calculation of a blood pressure value may be performed not only in the pressurization process but also in the depressurization process.

(Operation of Blood Pressure Measurement Based on Pulse Transit Time)

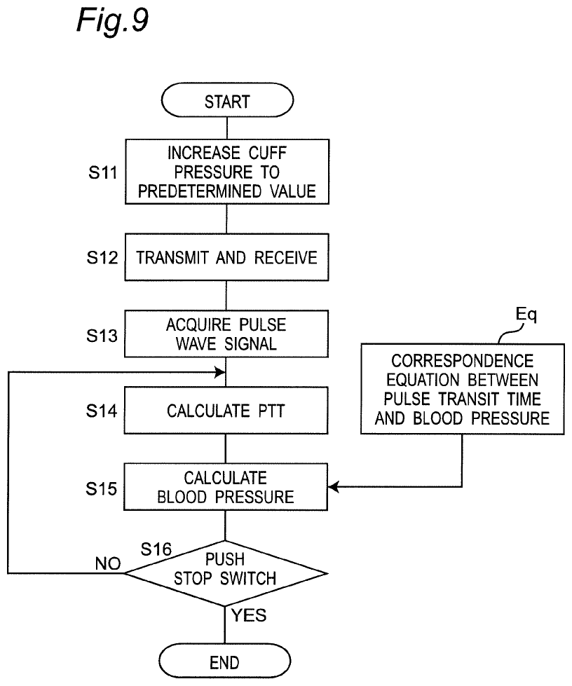

FIG. 9 shows an operation flow according to a pulse wave measurement method and a blood pressure measurement method of an exemplary embodiment of the present disclosure, in which the sphygmomanometer 1 performs pulse wave measurement to acquire a pulse transit time (PTT) and performs blood pressure measurement (estimation) based on the pulse transit time. The belt 20 of the sphygmomanometer 1 is assumed to be worn around the left wrist 90 in advance.

When the user instructs the blood pressure measurement based on the PTT using the push-type switch as the operation unit 52 installed on the main body 10, the CPU 100 starts operation. That is, the CPU 100 closes the valve 33 and drives the pump 32 via the pump drive circuit 320 to perform control of feeding air into the press cuff 21, which makes the press cuff 21 inflated and the cuff pressure Pc (see FIG. 6A) increased to a predetermined value (step S11 in FIG. 9). In this example, to reduce a physical burden on the user, the pressurization is curbed to such an extent (for example, about 5 mmHg) that the belt 20 can closely contact the left wrist 90. As a result, the transmitting/receiving antenna group 40E is surely in contact with the palm-side surface 90a of the left wrist 90, and thus no gap is generated between the palm-side surface 90a and the transmitting/receiving antenna group 40E. Note that step S11 may be omitted.

At this time, as shown in FIG. 6A, in each of the first pulse wave sensor 40-1 and the second pulse wave sensor 40-2, (a second surface 402b of) the dielectric layer 402 of the transmitting/receiving antenna group 40E is in contact with the palm-side surface 90a of the left wrist 90. Thus, in each of the first pulse wave sensor 40-1 and the second pulse wave sensor 40-2, the conductor layer 401 faces the palm-side surface 90a of the left wrist 90, and the dielectric layer 402 keeps the distance (distance in the thickness direction) between the palm-side surface 90a of the left wrist 90 and the conductor layer 401 constant. Further, as described above, the pair of transmitting/receiving antennas (41, 42) of the first set meets the upstream portion 91u of the radial artery 91 passing through the left wrist 90 in the longitudinal direction of the left wrist 90 (corresponding to the width direction Y of the belt 20). Meanwhile, the pair of transmitting/receiving antennas (44, 43) of the second set meets the downstream portion 91d of the radial artery 91.

Next, in this wearing state, as shown in step S12 in FIG. 9, the CPU 100 performs control of transmission and reception in each of the first pulse wave sensor 40-1 and the second pulse wave sensor 40-2 shown in FIG. 5. Specifically, as shown in FIG. 6A, in the first pulse wave sensor 40-1, the transmitting circuit 46 emits the radio wave E1 toward the upstream portion 91u of the radial artery 91 via the transmitting antenna 41, that is, from the conductor layer 401 through the dielectric layer 402 (or the gap present at the side of the dielectric layer 402). Further, the receiving circuit 47 receives the radio wave E1′ reflected from the upstream portion 91u of the radial artery 91 via the receiving antenna 42, that is, through the dielectric layer 402 (or the gap present at the side of the dielectric layer 402) by the conductor layer 401 to detect and amplify the received radio wave. In the second pulse wave sensor 40-2, the transmitting circuit 49 emits the radio wave E2 toward the downstream portion 91d of the radial artery 91 via the transmitting antenna 44, that is, from the conductor layer 401 through the dielectric layer 402 (or the gap present at the side of the dielectric layer 402). Further, the receiving circuit 48 receives the radio wave E2′ reflected from the downstream portion 91d of the radial artery 91 via the receiving antenna 43, that is, through the dielectric layer 402 (or the gap present at the side of the dielectric layer 402) by the conductor layer 401 to detect and amplify the received radio wave. In this example, the radio wave E1 emitted in the first pulse wave sensor 40-1 and the radio wave E2 emitted in the second pulse wave sensor 40-2 have a bandwidth narrowed by a predetermined bandwidth index (the bandwidth will be described in detail later).

Next, as shown in step S13 in FIG. 9, the CPU 100 works as the pulse wave detector 101, 102 to acquire the pulse wave signal PS1, PS2 as shown in FIG. 6B in each of the first pulse wave sensor 40-1 and the second pulse wave sensor 40-2 shown in FIG. 5. That is, in the first pulse wave sensor 40-1, the CPU 100 works as the pulse wave detector 101 to acquire the pulse wave signal PS1 representing the pulse wave at the upstream portion 91u of the radial artery 91 from outputs of the receiving circuit 47 when the blood vessel expands and when contracts. In the second pulse wave sensor 40-2, the CPU 100 works as the pulse wave detector 102 to acquire the pulse wave signal PS2 representing the pulse wave at the downstream portion 91d of the radial artery 91 from outputs of the receiving circuit 48 when the blood vessel expands and when contracts.

Next, as shown in step S14 in FIG. 9, the CPU 100 works as the PTT calculator 103 as the time difference acquisition unit to acquire the time difference between the pulse wave signal PS1 and the pulse wave signal PS2 as the pulse transit time (PTT). More specifically, in this example, the CPU 100 acquires the time difference Δt between the peak A1 of the first pulse wave signal PS1 and the peak A2 of the second pulse wave signal PS2 shown in FIG. 6B as the pulse transit time (PTT).

Then, as shown in step S15 in FIG. 9, the CPU 100 works as the first blood pressure calculator to calculate (estimate) the blood pressure based on the pulse transit time (PTT) acquired in step S14 using a predetermined correspondence equation Eq between pulse transit time and blood pressure. Here, when the pulse transit time is represented by DT and the blood pressure is represented by EBP, the predetermined correspondence equation Eq between pulse transit time and blood pressure is provided as a publicly-known fractional function including a term of 1/DT² as shown by, for example,

EBP=α/DT²+β (Eq. 1)

(where, each of α and β represents a known coefficient or a constant)

(see, for example, JP H10-201724 A). Alternatively, as the predetermined correspondence equation Eq between pulse transit time and blood pressure, another publicly-known correspondence equation including, in addition to the term of 1/DT², a term of 1/DT and a term of DT, such as

EBP=α/DT²+β/DT+γDT+δ (Eq. 2)

(where, each of α, β, γ, and δ represents a known coefficient or a constant)

may be used.

When the blood pressure is calculated (estimated) in this manner as described above, in each of the first pulse wave sensor 40-1 and the second pulse wave sensor 40-2, the dielectric layer 402 keeps the distance between the palm-side surface 90a of the left wrist 90 and the conductor layer 401 constant. Further, the interposition of the dielectric layer 402 between the palm-side surface 90a of the left wrist 90 and the conductor layer 401 results in suppressing influence of permittivity variation of a living body (relative permittivity of a living body varies in a range of about 5 to 40). Further, since the distance between the palm-side surface 90a of the left wrist 90 and the conductor layer 401 can be secured, a range (area) at which the radio wave is aimed on the palm-side surface 90a of the left wrist 90 can be increased as compared with the case where the conductor layer 401 is in direct contact with the palm-side surface 90a of the left wrist 90. Accordingly, even when the wearing position of the conductor layer 401 is slightly misaligned from right above the radial artery 91, the signal reflected from the radial artery 91 can be stably received. As a result, signal levels received by the receiving circuits 47, 48 are stable, and thus the pulse wave signals PS1, PS2 as biological information can be acquired with high accuracy. This allows for acquiring the pulse transit time (PTT) with high accuracy, and thus calculating (estimating) the blood pressure value with high accuracy. The measurement result of the blood pressure value is displayed on the display unit 50 and recorded in the memory 51.

In this example, in step S16 in FIG. 9, when measurement stop is not instructed using the push-type switch as the operation unit 52 (NO in step S16), the pulse transit time (PTT) calculation (step S14 in FIG. 9) and the blood pressure calculation (estimation) (step S15 in FIG. 9) are periodically repeated each time the first and second pulse wave signals PS1, PS2 depending on the pulse waves are input. The CPU 100 updates and displays the measurement result of the blood pressure value on the display unit 50, and stores and records the measurement result in the memory 51. In step S16 in FIG. 9, when the measurement stop is instructed (YES in step S16), the measurement operation ends.

According to the sphygmomanometer 1, the blood pressure measurement based on the pulse transit time (PTT) allows for continuous measurement of blood pressure over a long duration with a light physical burden on the user.

Further, according to the sphygmomanometer 1, the single device can perform the blood pressure measurement (estimation) based on the pulse transit time and the blood pressure measurement by the oscillometric method using the common belt 20. Therefore, user convenience can be enhanced. For example, in general, when the blood pressure measurement (estimation) based on the pulse transit time (PTT) is performed, calibration of the correspondence equation Eq between pulse transit time and blood pressure (in the above example, update of the values such as the coefficients α, β based on the pulse transit time and the blood pressure value that have been measured) is appropriately required. Here, according to the sphygmomanometer 1, the single device can perform the blood pressure measurement by the oscillometric method and calibrate the correspondence equation Eq based on the result. Therefore, user convenience can be enhanced. Further, a rapid rise in blood pressure can be captured by the PTT method (blood pressure measurement based on the pulse transit time) that allows for continuous measurement with low accuracy. The rapid rise in blood pressure can be used as a trigger to start the measurement by the oscillometric method with higher accuracy.

(Bandwidth of Radio Waves E1, E2 Emitted in First Pulse Wave Sensor 40-1 and Second Pulse Wave Sensor 40-2)

Assuming that the radio waves E1, E2 emitted in the first pulse wave sensor 40-1 and the second pulse wave sensor 40-2 include high-order wide frequency components as included in a square wave (pulse wave), the received radio waves E1′, E2′ also include high-order wide frequency components. Consequently, there arises a problem that the pulse wave detectors 101, 102 have to perform complicated signal processing such as the Fourier transform.

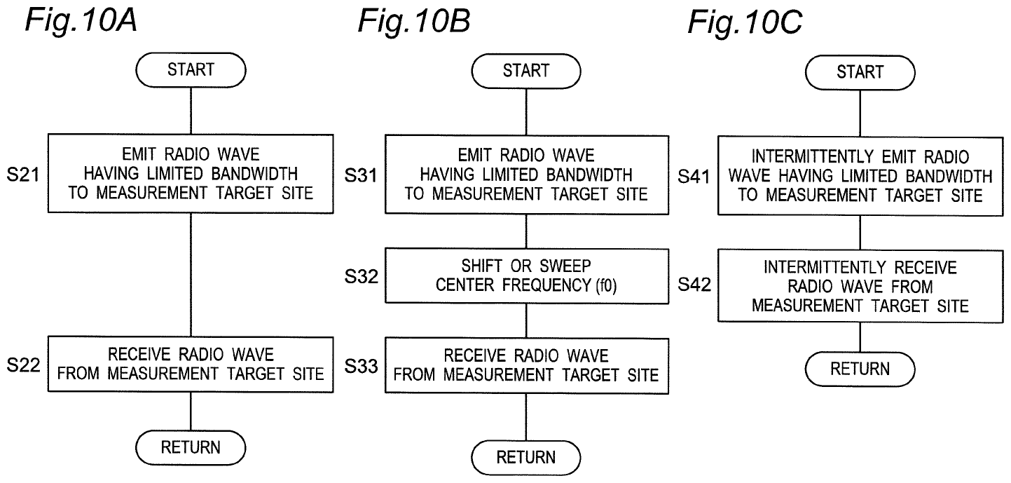

Thus, in the sphygmomanometer 1, an operation flow in FIG. 10A is performed in step S12 of performing transmission and reception in FIG. 9. Specifically, as shown in step S21, the transmitters 61, 64 respectively emit the radio waves E1, E2 having the bandwidth narrowed by the predetermined bandwidth index toward the upstream portion 91u and the downstream portion 91d of the radial artery 91 (hereinafter referred to as “measurement target sites 91u, 91d”). The processing proceeds to step 22, and the receivers 62, 63 receive the radio waves E1′, E2′ having the narrowed bandwidth from the measurement target sites. Then, the processing returns to the main flow (FIG. 9). In this example, the “bandwidth index” refers to an occupied bandwidth representing a range occupied by radio wave frequencies, a fractional bandwidth obtained by dividing the occupied bandwidth by a center frequency (f₀) (=occupied bandwidth/center frequency (f₀)), or the like. When the “fractional bandwidth” (represented by reference sign RBW) is used as the “bandwidth index”, the fractional bandwidth RBW is preferably 0.03 or smaller.

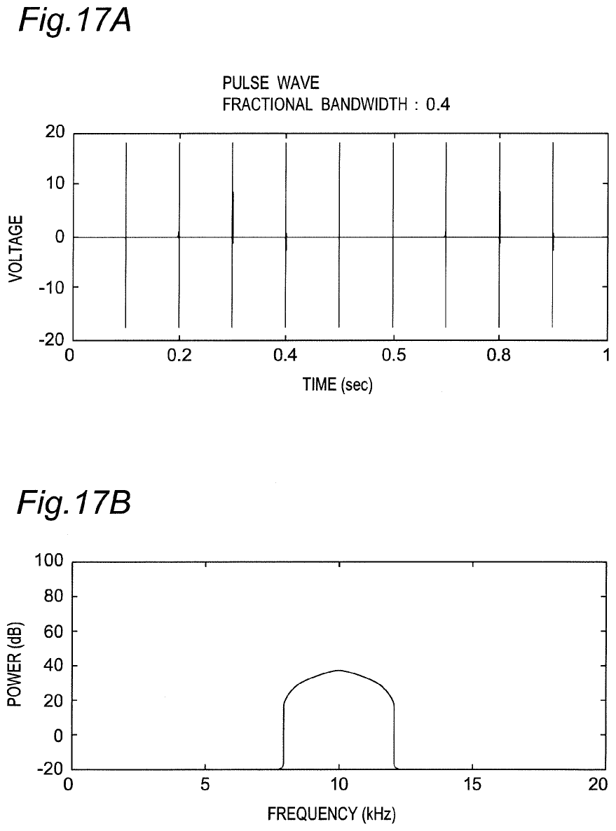

In the sphygmomanometer 1, the radio waves E1, E2 emitted from the transmitters 61, 64 have the bandwidth narrowed by the predetermined bandwidth index, and thus do not include wide frequency components as included in a square wave. Accordingly, the outputs of the receivers 62, 63 that receive the radio waves E1′, E2′ reflected from the measurement target sites 91u, 91d do not include wide frequency components as included in a square wave. Therefore, when the pulse wave detectors 101, 102 detect, based on the outputs of the receivers 62, 63, the pulse wave signals PS1, PS2 representing the pulse waves at measurement target sites 91u, 91d, it is possible to obtain the pulse wave signals PS1, PS2 having a high S/N ratio without requiring complicated signal processing such as the Fourier transform. That is, the pulse wave signals PS1, PS2 can be acquired with high accuracy. Note that a pulse-shaped square wave as shown in FIG. 17A (in this example, the center frequency is 10 kHz) includes wide frequency components (in this example, the fractional bandwidth is 0.4) as shown in FIG. 17B.

In calculation of the S/N ratio, as the signal (S), the amplitude or the standard deviation of the pulse wave signal PS1, PS2 when the radio wave is transmitted during wearing on a human body (in this example, the left wrist 90) is used. As the noise (N), the amplitude or the standard deviation of the pulse wave signal PS1, PS2 when no radio wave is emitted during wearing on the human body is used. Alternatively, the amplitude or the standard deviation of the pulse wave signal PS1, PS2 when the radio wave is emitted during non-wearing on the human body is used.

Here, the sphygmomanometer 1 includes the first pulse wave sensor 40-1 and the second pulse wave sensor 40-2 as shown in FIG. 5. However, the first pulse wave sensor 40-1 or the second pulse wave sensor 40-2 alone may be included as the pulse wave sensor. Hereinafter, the first pulse wave sensor 40-1 and the second pulse wave sensor 40-2 are collectively referred to as “pulse wave sensors 40-1, 40-2”.

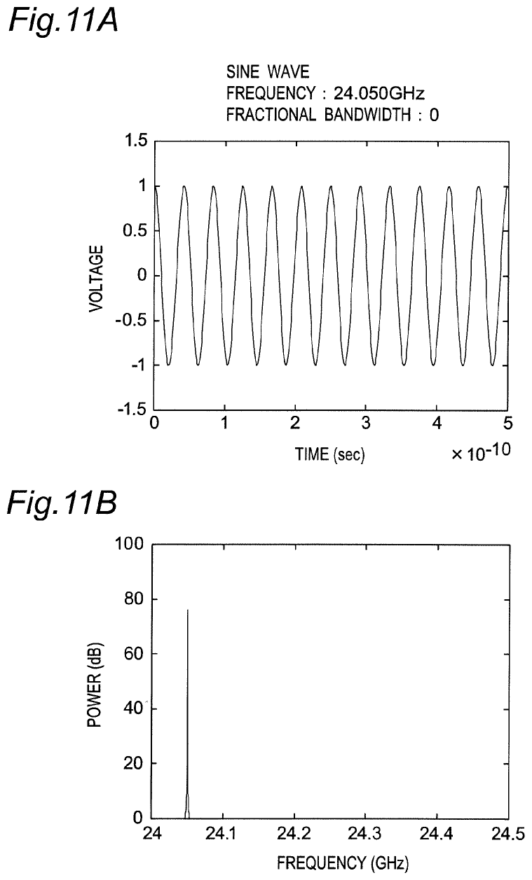

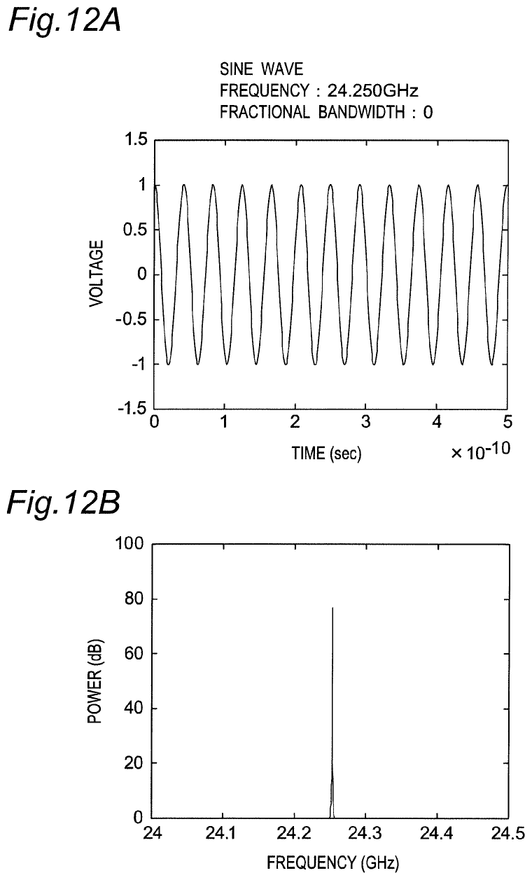

An example of the radio waves E1, E2 having the bandwidth narrowed by the above-described predetermined bandwidth index is a continuous wave (CW) as shown in FIGS. 11 (A) and 12 (A). The continuous waves typically include a sine wave.

(Example of Continuous Sine Wave)

In an example in FIG. 11A, the frequency of a sine wave is 24.050 GHz. The amplitude of this sine wave is 1.0 V. FIG. 11B shows a frequency spectrum according to this example. In this example, the frequency spectrum does not include wide frequency components and has a linear rise at the center frequency of 24.050 GHz. The power is about 80 dB. In this example, the fractional bandwidth RBW is logically zero.

In this example, in step S21 in FIG. 10A, the transmitters 61, 64 continuously emit the radio waves E1, E2 having the narrowed bandwidth to the measurement target sites 91u, 91d. In step S22, the receiver 62 continuously receives the radio waves E1′, E2′ from the measurement target sites.

FIG. 12A shows an example of a sine wave having a frequency different from the frequency of the example in FIG. 11A. In this example, the frequency of the sine wave is 24.250 GHz. The amplitude of this sine wave is 1.0 V. FIG. 12B shows a frequency spectrum according to this example. In this example, the frequency spectrum does not include wide frequency components and has a linear rise at a center frequency of 24.250 GHz. The power is about 80 dB. In this example, the fractional bandwidth RBW is logically zero.

In this example, an operation flow in FIG. 10B is especially performed in step S12 of performing transmission and reception in FIG. 9 described above. Specifically, as shown in step S31, the transmitters 61, 64 emit the radio waves E1, E2 having the narrowed bandwidth to the measurement target sites 91u, 91d. The processing proceeds to step S32, and the transmitter 61 shifts or sweeps the center frequency (f₀) of the radio wave. The processing proceeds to step S33, and the receiver 62 receives the radio waves E1′, E2′ from the measurement target sites. Then, the processing returns to the main flow (FIG. 9). Here, the transmitters 61, 64 shift or sweep the center frequency (f₀) by 200 MHz from 24.050 GHz to 24.250 GHz. When the shift or sweep is performed in this manner, for example, the pulse wave sensors 40-1, 40-2 measure the pulse wave signals PS1, PS2 for 10 seconds. If the S/N ratio of the pulse wave signals PS1, PS2 is smaller than a predetermined threshold (represented by α), the transmitters 61, 64 shift or sweep the frequency to a next candidate frequency (described in detail later).

In this example, the transmitters 61, 64 shift or sweep the center frequency (f₀) of the radio waves E1, E2 having the narrowed bandwidth. Accordingly, even when the measurement is difficult at a specific frequency due to an individual difference of human body composition, another frequency obtained by shifting or sweeping the frequency can be used. As a result, it is more likely that the pulse wave signals PS1, PS2 can be acquired with high accuracy.

(Example of Intermittent Sine Wave)

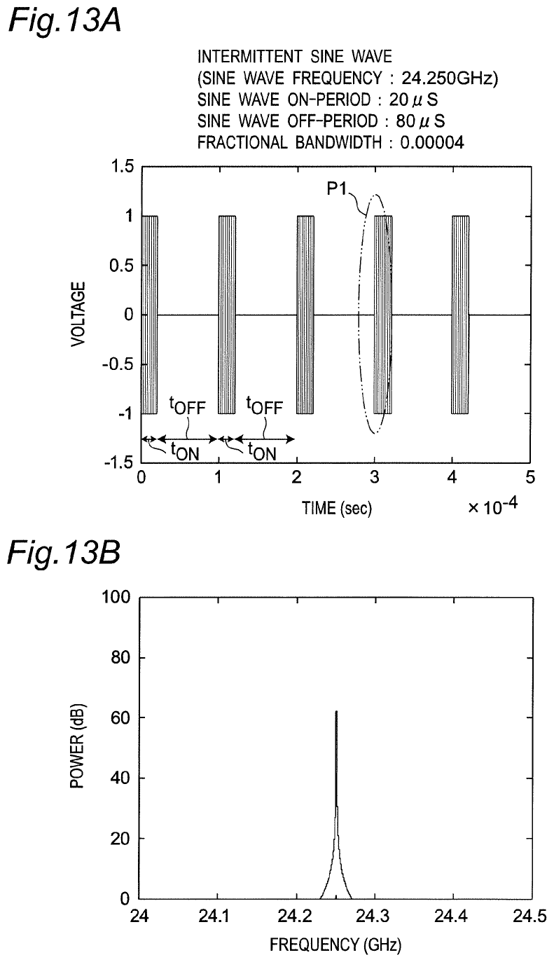

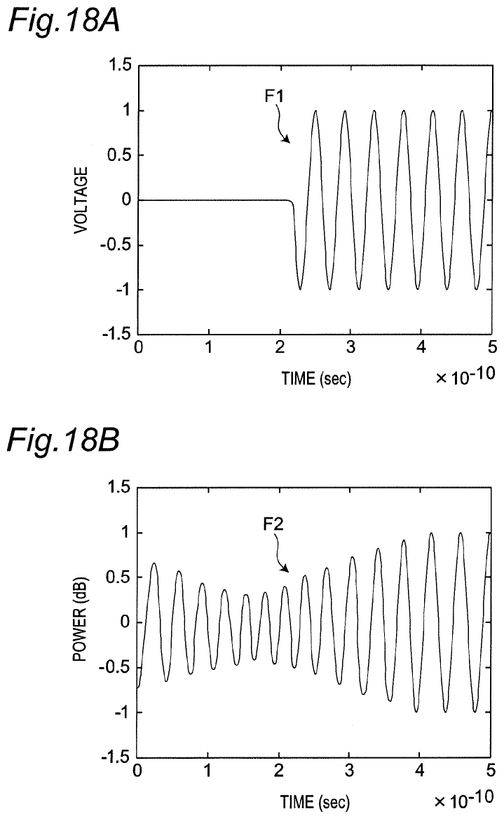

FIG. 13A shows an example of an intermittent sine wave that repeats an on-period t_{ON} and an off-period t_{OFF}. In this example, the frequency of the sine wave is 24.250 GHz. The amplitude of this sine wave is 1.0 V. This example shows the intermittent sine wave having the sine wave on-period t_{ON} of 20 microseconds and the sine wave off-period t_{OFF} of 80 microseconds. A partial schematic diagram of this waveform within a range surrounded by a two-dot chain line P1 is shown in FIG. 18A. FIG. 18A is the partial schematic diagram of the intermittent sine wave F1 that is in the off-period t_{OFF} and then the on-period t_{ON}. FIG. 13B shows a frequency spectrum according to the example of this intermittent sine wave. In this example, the frequency spectrum does not include wide frequency components and has a symmetrical triangular rise around a center frequency of 24.250 GHz. The power is about 60 dB at the center frequency. In this example, the fractional bandwidth RBW is 0.00004.

In this example, an operation flow in FIG. 10C is especially performed in step 12 of performing transmission and reception in FIG. 9 described above. Specifically, as shown in step S41, the transmitters 61, 64 intermittently emit the radio waves E1, E2 having the narrowed bandwidth to the measurement target sites 91u, 91d. The processing proceeds to step S42, and the receivers 62, 63 intermittently receive the radio waves E1′, E2′ from the measurement target sites. Then, the processing returns to the main flow (FIG. 9).

In this example, the transmitters 61, 64 intermittently transmit the radio waves E1, E2 having the narrowed bandwidth. Accordingly, the receivers 62, 63 intermittently receive the radio waves E1′, E2′ reflected from the measurement target sites 91u, 91d. Therefore, the power consumption of the transmitters 61, 64 and the receivers 62, 63 is reduced and the power consumption of the pulse wave detectors 101, 102 is also reduced as compared with the case of the continuous transmission and reception. Here, for example, the power consumption in the case of the continuous transmission is 155.1 mWh. Comparatively, the power consumption in the case of the intermittent transmission (for example, duty ratio is 1%) is reduced to 6.5 mWh.

(Example of Modulated Wave)

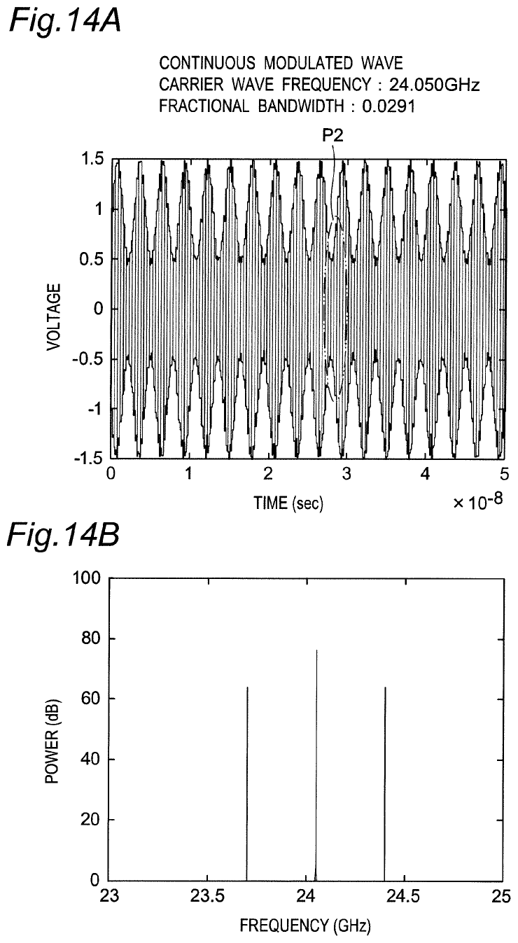

FIG. 14A shows an example of a continuous modulated wave created by superimposing a modulating signal wave on a carrier wave. In this example, the frequency of the carrier wave is 24.050 GHz. The amplitude of this modulated wave is 1.5 V. In this example, the modulation method is amplitude modulation. The frequency of the modulating signal wave is 350 MHz, and the modulation degree is 0.5. A partial schematic diagram of this waveform within a range surrounded by a two-dot chain line P2 is shown in FIG. 18B. FIG. 18B shows the partial schematic diagram of the continuous modulated wave F2. FIG. 14B shows a frequency spectrum according to this continuous modulated wave. In this example, the frequency spectrum does not include wide frequency components, has a linear rise around a center frequency of 24.050, and includes a lower side band (LSB) and an upper side band (USB) on the left and right of the center frequency. The power is about 80 dB at the center frequency. In this example, the fractional bandwidth RBW is 0.0291.

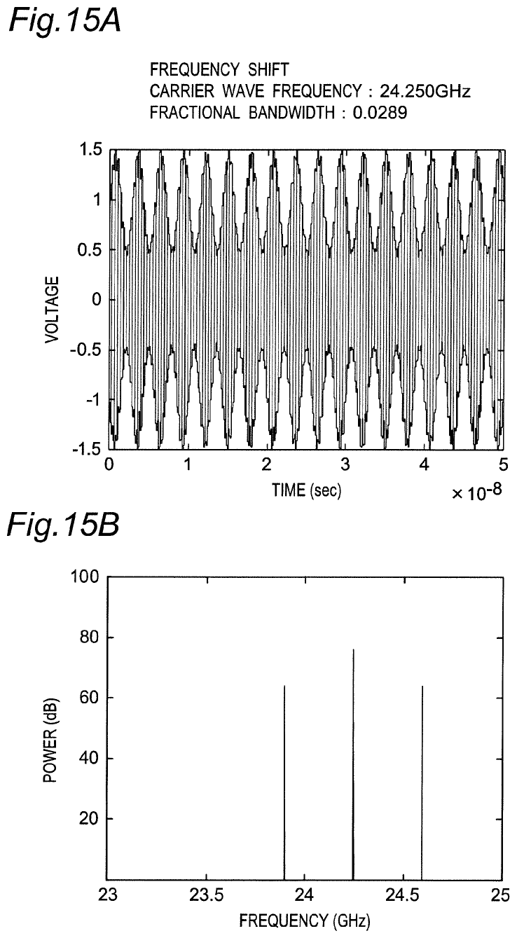

FIG. 15A shows an example of a modulated wave having a frequency different from the frequency of the example in FIG. 14A. In this example, the frequency of the carrier wave is 24.250 GHz. The amplitude of this modulated wave is 1.5 V. In this example, the modulation method is amplitude modulation. The frequency of the modulating signal wave is 350 MHz, and the modulation degree is 0.5. FIG. 15B shows a frequency spectrum according to this continuous modulated wave. In this example, the frequency spectrum does not include wide frequency components, has a linear rise around a center frequency of 24.250 GHz, and includes a lower side band (LSB) and an upper side band (USB) on the left and right of the center frequency. The power is about 80 dB at the center frequency. In this example, the fractional bandwidth RBW is 0.0289.

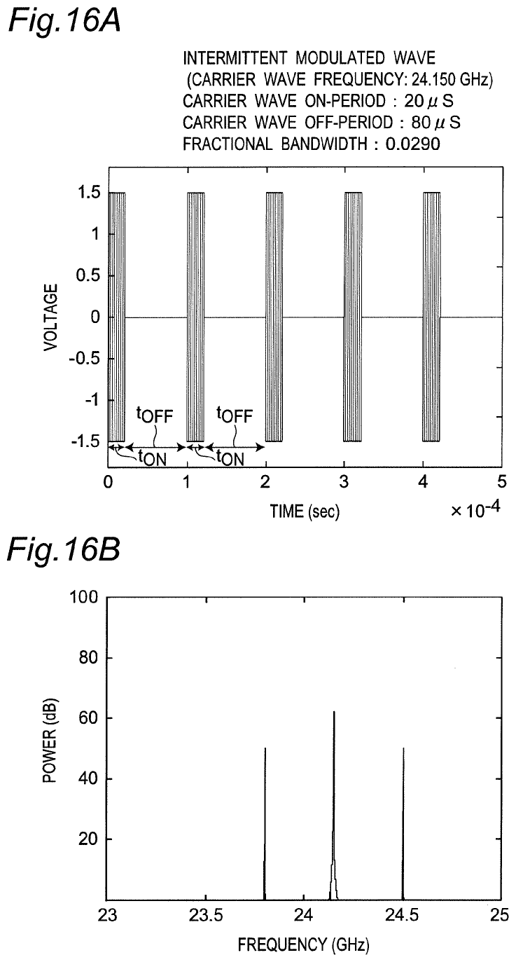

FIG. 16A shows an example of an intermittent modulated wave that repeats an on-period t_{ON} and an off-period t_{OFF}. In this example, the frequency of the carrier wave is 24.150 GHz. The amplitude of this modulated wave is 1.5 V. In this example, the modulation method is amplitude modulation. The frequency of the signal wave is 350 MHz, and the modulation degree is 0.5. This example shows the intermittent modulated wave having the carrier wave on-period t_{ON} of 20 microseconds and the carrier wave off-period t_{OFF} of 80 microseconds. FIG. 16B shows a frequency spectrum according to this intermittent modulated wave. In this example, the frequency spectrum does not include wide frequency components, has a linear rise around a center frequency of 24.150 GHz, and includes a lower side band (LSB) and an upper side band (USB) on the left and right of the center frequency. The power is about 60 dB at the center frequency. In this example, the fractional bandwidth RBW is 0.0290.

As shown in FIGS. 11 to 16, in the pulse wave sensors 40-1, 40-2, the radio waves E1, E2 emitted from the transmitters 61, 64 have the bandwidth narrowed by the predetermined bandwidth index. Specifically, the fractional bandwidth RBW is narrowed to 0.03 or smaller. Such radio waves E1, E2 do not include wide frequency components (see FIG. 17B) as included in the square wave (pulse wave) shown in FIG. 17A. Accordingly, the outputs of the receivers 62, 63 that receive the radio waves E1′, E2′ reflected from the measurement target sites 91u, 91d do not include wide frequency components as included in the square wave (pulse wave). Therefore, when the pulse wave detectors 101, 102 detect, based on the outputs of the receivers 62, 63, pulse wave signals PS1, PS2 representing the pulse waves of the artery passing through the measurement target sites 91u, 91d, it is possible to obtain the pulse wave signals PS1, PS2 having a high S/N ratio without requiring complicated signal processing such as the Fourier transform.

(Method for Switching and Shifting Frequency Based on Signal-to-Noise Ratio of Pulse Wave Signal)

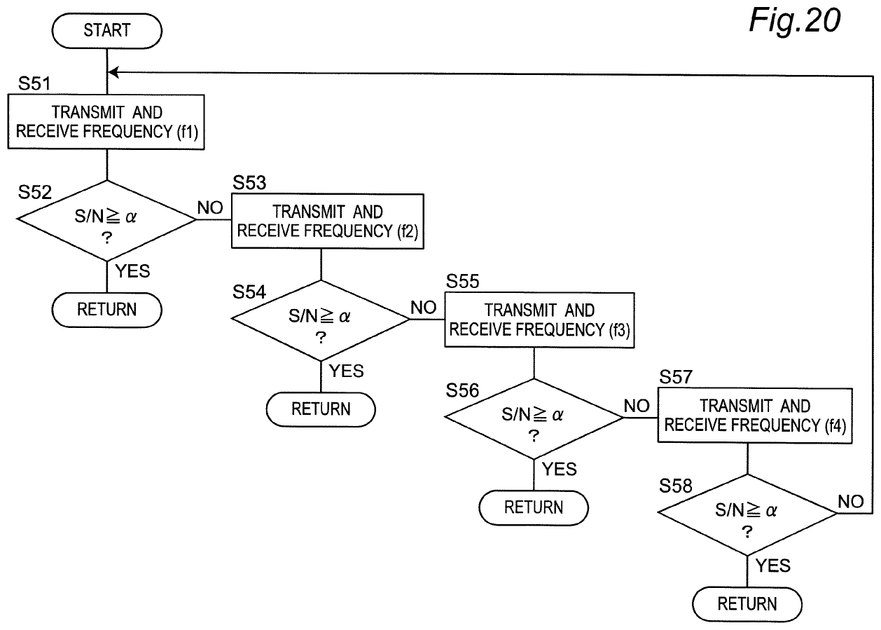

FIG. 20 shows another flow of control that causes the transmitters 61, 64, while performing transmission and reception in step S12 in FIG. 9 described above, to switch and shift the frequency.

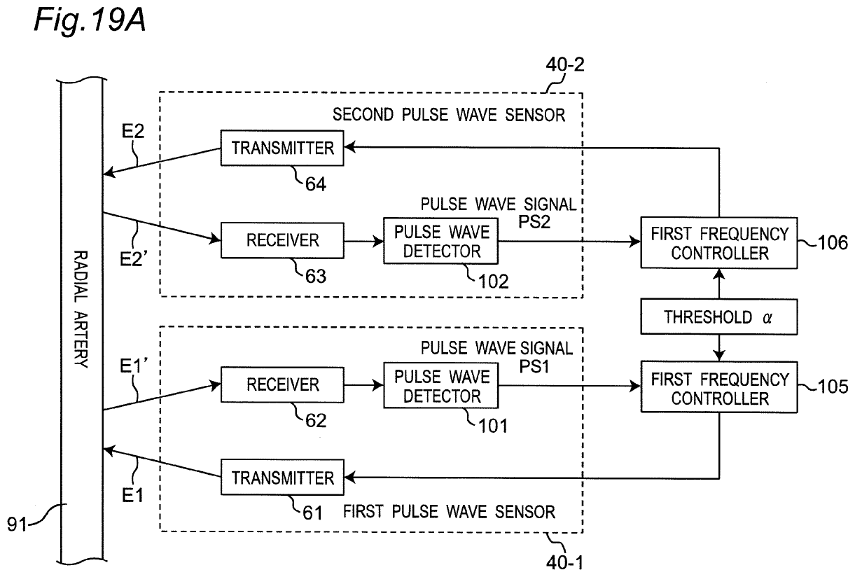

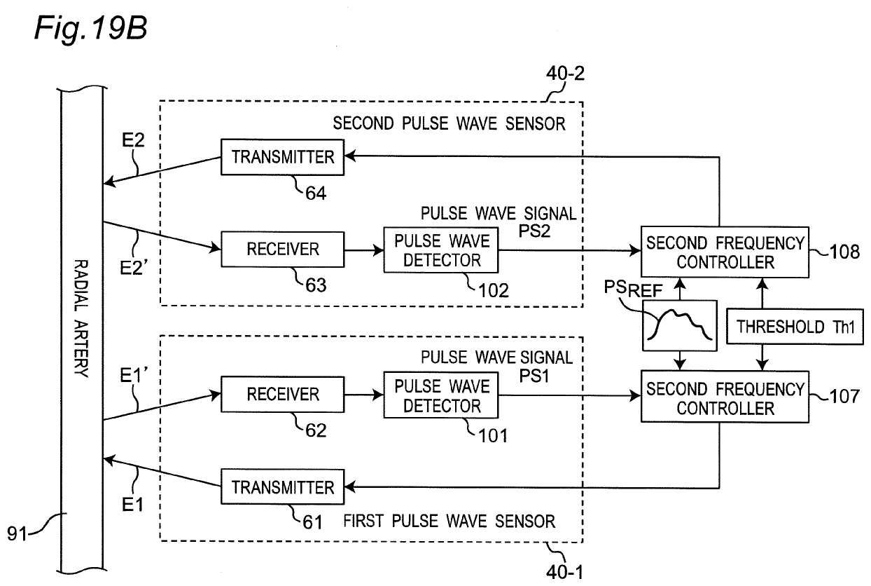

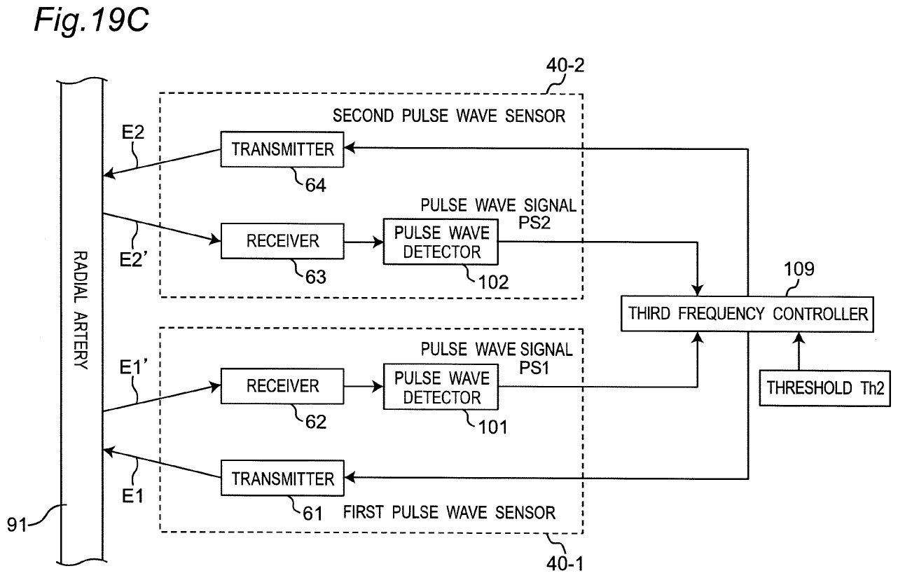

FIG. 19A shows a block configuration implemented by a program for performing processing according to the flow in FIG. 20 in the sphygmomanometer 1. In this block configuration, first frequency controllers 105, 106 are implemented corresponding to the pulse wave sensors 40-1, 40-2, respectively.

In this example, the first frequency controllers 105, 106 acquire the signal-to-noise ratio (S/N) of the pulse wave signals PS1, PS2, respectively, and determine whether the acquired S/N is larger than a threshold α as a reference value (in this example, predetermined at α=40 dB and stored in the memory 51). If the signal-to-noise ratio (S/N) of the pulse wave signals PS1, PS2 is S/N≥α, the first frequency controllers 105, 106 determine that the frequency is appropriate, respectively. If the signal-to-noise ratio (S/N) of the pulse wave signals PS1, PS2 is S/N<α, the first frequency controllers 105, 106 determine that the frequency is inappropriate, and perform control that causes the corresponding transmitters 61, 64 to switch and shift the frequency.

For example, processing by the first frequency controller 105 in the pulse wave sensor 40-1 will be described using the flow in FIG. 20.

In this example, first, as shown in step S51 in FIG. 20, the first frequency controller 105 selects a frequency (f₁) among frequencies (f₁), (f₂), (f₃), (f₄). In response to this selection, the transmitter 61 emits a radio wave having the frequency (f₁). As a result, the pulse wave detector 101 acquires the signal-to-noise ratio (S/N) of the pulse wave signal PS1 representing the pulse wave of the radial artery 91.

Next, as shown in step S52 in FIG. 20, the first frequency controller 105 acquires the signal-to-noise ratio (S/N) of the pulse wave signal PS1, PS2, and determines whether the acquired S/N is larger than the threshold α as the reference value. Here, if the signal-to-noise ratio (S/N) of the pulse wave signal PS1 is S/N≥α (YES in step S52), it is determined that the current frequency (f₁) is appropriate, and the processing returns to the men-in flow (FIG. 9).

Meanwhile, in step S52 in FIG. 20, if the signal-to-noise ratio (S/N) of the pulse wave signal PS1 is S/N<α (NO in step S52), the processing proceeds to step S53, and the first frequency controller 105 selects the frequency (f₂) among the frequencies (f₁), (f₂), (f₃), (f₄). In response to this selection, the transmitter 61 emits a radio wave having the frequency (f₂). As a result, the pulse wave detector 101 acquires the pulse wave signal PS1.

Next, as shown in step S54 in FIG. 20, the first frequency controller 105 acquires the signal-to-noise ratio (S/N) of the pulse wave signal PS1, and determines whether the acquired S/N is larger than the threshold α. Here, if the signal-to-noise ratio (S/N) of the pulse wave signal PS1 is S/N≥α (YES in step S54), it is determined that the current frequency (f₂) is appropriate, and the processing returns to the men-in flow (FIG. 9).

Meanwhile, in step S54 in FIG. 20, if the signal-to-noise ratio (S/N) of the pulse wave signal PS1 is S/N<α (NO in step S54), the processing proceeds to step S55, and the first frequency controller 105 selects the frequency (f₃) among the frequencies (f₁), (f₂), (f₃), (f₄). In response to this selection, the transmitter 61 emits a radio wave having the frequency (f₃). As a result, the pulse wave detector 101 acquires the pulse wave signal PS1.

Next, as shown in step S56 in FIG. 20, the first frequency controller 105 acquires the signal-to-noise ratio (S/N) of the pulse wave signal PS1, and determines whether the acquired S/N is larger than the threshold α as the reference value. Here, if the signal-to-noise ratio (S/N) of the pulse wave signal PS1 is S/N≥α (YES in step S56), it is determined that the current frequency (f₃) is appropriate, and the processing returns to the men-in flow (FIG. 9).

Meanwhile, in step S56 in FIG. 20, if the signal-to-noise ratio (S/N) of the pulse wave signal PS1 is S/N<α (NO in step S56), the processing proceeds to step S57, and the first frequency controller 105 selects the frequency (f₄) among the frequencies (f₁), (f₂), (f₃), (f₄). In response to this selection, the transmitter 61 emits a radio wave having the frequency (f₄). As a result, the pulse wave detector 101 acquires the pulse wave signal PS1.

Next, as shown in step S58 in FIG. 20, the first frequency controller 105 acquires the signal-to-noise ratio (S/N) of the pulse wave signal PS1, and determines whether the acquired S/N is larger than the threshold α as the reference value. Here, if S/N≥α (YES in step S58), it is determined that the current frequency is appropriate, and the processing returns to the men-in flow (FIG. 9).

Meanwhile, in step S58 in FIG. 20, if the pulse wave signal PS1 has S/N<α (NO in step S58), the processing returns to step S51 and is repeated. Note that, when no frequency appropriate for use is found even after the processing of steps S51 to S58 in FIG. 20 is repeated a predetermined number of times, or no frequency appropriate for use is found even after a predetermined period has elapsed, in this embodiment, the CPU 100 causes an error display to appear on the display unit 50 and terminates the processing. This makes it possible to surely and quickly determine a frequency appropriate for use among the plurality of frequencies (f₁), (f₂), (f₃), (f₄).

The first frequency controller 106 in the pulse wave sensor 40-2 also performs the same processing as the flow in FIG. 20.

In this manner, when a frequency appropriate for use is selected according to the flow in FIG. 20, the transmitters 61, 64 respectively emit the radio waves E1, E2 having the selected frequency. As a result, the pulse wave detectors 101, 102 can obtain the pulse wave signals PS1, PS2 having a high S/N ratio.

(Method for Shifting or Sweeping Frequency Based on Cross-Correlation Coefficient Between Waveform of Pulse Wave Signal and Reference Waveform)

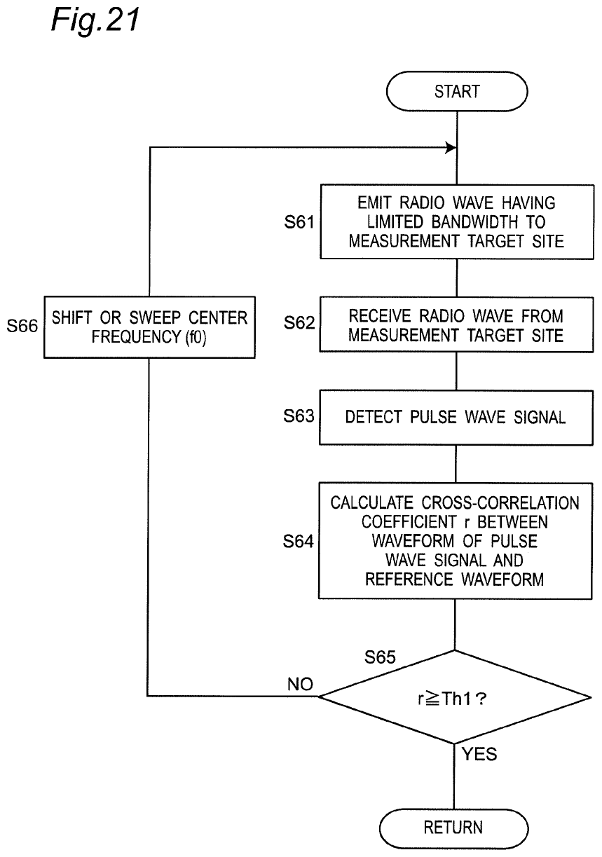

FIG. 21 shows a flow of another control that causes the transmitters 61, 64, while performing transmission and reception in step S12 in FIG. 9 described above, to shift or sweep the frequency based on the cross-correlation coefficient (represented by reference sign r) between the waveform of the pulse wave signal output in a time series manner by the pulse wave detectors 101, 102 of the pulse wave measurement devices and a reference waveform.

FIG. 19B shows a block configuration implemented by a program for performing processing according to the flow in FIG. 21 in the sphygmomanometer 1. In this block configuration, second frequency controllers 107, 108 are implemented.

In this example, the second frequency controllers 107, 108 shown in FIG. 19B calculate in real time the cross-correlation coefficient r between the waveform of the pulse wave signal output in a time series manner by the pulse wave detectors 101, 102 and a predetermined reference waveform PS_{REF}, respectively. Then, the second frequency controllers 107, 108 determine whether the calculated cross-correlation coefficient r exceeds a predetermined threshold Th1 (in this example, predetermined at Th1=0.99 and stored in the memory 51), and perform control that causes the transmitters 61, 64 to shift or sweep the center frequency (f₀) to make the cross-correlation coefficient r equal to or larger than the threshold Th1.

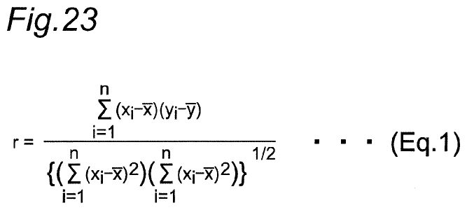

In this example, when two sets of data string {xi} and data string {yi} (where i=1, 2, . . . , n) consisting of numerical values are given, the cross-correlation coefficient r between the data string {xi} and the data string {yi} is defined by the equation (Eq. 1) shown in FIG. 23. In the equation (Eq. 1), x and y with an upper bar represent average values of x and y, respectively.

As the reference waveform PS_{REF}, an output waveform when the pulse wave detectors 101, 102 normally detect the pulse wave signals PS1, PS2 having a high S/N ratio is set in advance. The reference waveform PS_{REF} is stored in the memory 51.

For example, processing by the second frequency controller 107 in the pulse wave sensor 40-1 will be described using the flow in FIG. 21.

First, as shown in step S61 in FIG. 21, the transmitter 61, 64 emits the radio wave having the narrowed bandwidth to the measurement target site. Accordingly, as shown in step S62, the receiver 62, 63 receives the radio wave from the measurement target site 91u, 91d. The processing proceeds to step S63, and the pulse wave detector 101, 102 detects the pulse wave signal PS1, PS2.

Next, as shown in step S64 in FIG. 21, the second frequency controller 107 calculates in real time the cross-correlation coefficient r between the waveform of the pulse wave signal PS1 output in a time series manner by the pulse wave detector 101, 102 of the pulse wave measurement device and the reference waveform PS_{REF}. Furthermore, the second frequency controller 107 determines whether the calculated cross-correlation coefficient r exceeds the predetermined threshold Th1 (=0.99) (step S65 in FIG. 21). Here, if any of the cross-correlation coefficients r calculated by the frequency controllers 105, 106 is equal to or smaller than the threshold Th1 (NO in step S65 in FIG. 21), the processing of steps S61 to S65 is repeated until the cross-correlation coefficients r both exceed the threshold Th1. When the cross-correlation coefficients r calculated by the frequency controllers 105, 106 both exceed the threshold Th1 (YES in step S65 in FIG. 21), it is determined that the frequency is appropriate, and the processing returns to the men-in flow (FIG. 9).

The second frequency controller 108 in the pulse wave sensor 40-2 also performs the same processing as the flow in FIG. 21.

In this manner, when a frequency appropriate for use is selected according to the flow in FIG. 21, the transmitters 61, 64 respectively emit the radio waves E1, E2 having the selected frequency. In this example, the similarity between the output waveform of the pulse wave detectors 101, 102 and the reference waveform PS_{REF} is increased. As a result, the pulse wave detectors 101, 102 can obtain the pulse wave signals PS1, PS2 having a high S/N ratio.

(Method for Shifting or Sweeping Frequency Based on Cross-Correlation Coefficient Between Output Waveform of First Pulse Wave Signal and Output Waveform of Second Pulse Wave Signal)

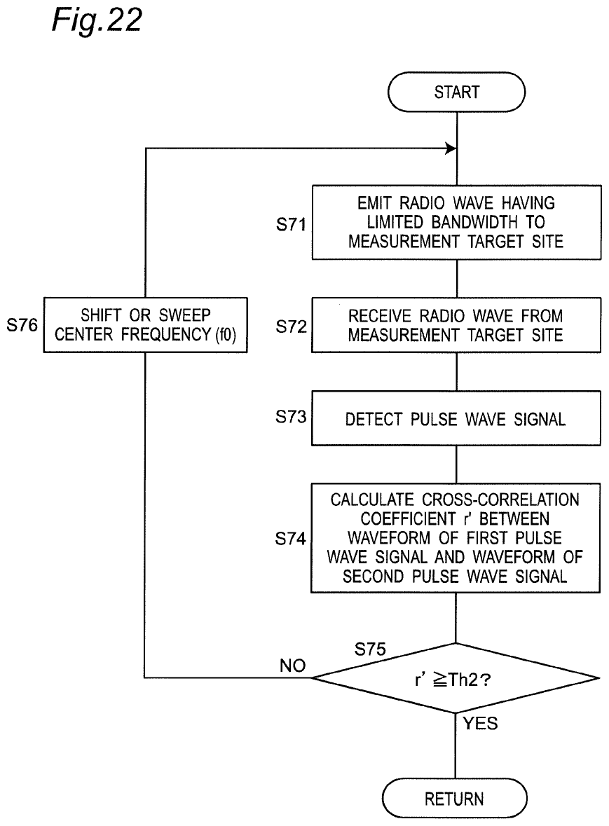

FIG. 22 shows a flow of another control that causes the transmitters 61, 64, while performing transmission and reception in step S12 in FIG. 9 described above, to shift or sweep the frequency based on the cross-correlation coefficient (represented by reference sign r′ and defined by the equation (Eq. 1) shown in FIG. 23 as with the above-described cross-correlation coefficient r) between the output waveform of the pulse wave signal PS1 output by the pulse wave detector 101 and the output waveform of the pulse wave signal PS2 output by the pulse wave detector 102.

FIG. 19C shows a block configuration implemented by a program for performing processing according to the flow in FIG. 22 in the sphygmomanometer 1. In this block configuration, a third frequency controller 109 is implemented.

In this example, the third frequency controller 109 calculates in real time the cross-correlation coefficient r′ between the output waveform of the pulse wave signal PS1 output by the pulse wave detector 101 and the output waveform of the pulse wave signal PS2 output by the pulse wave detector 102. The third frequency controller 109 also determines whether the calculated cross-correlation coefficient r′ exceeds a predetermined threshold Th2 (in this example, predetermined at Th2=0.99 and stored in the memory 51), and performs control that causes the transmitter 61 or 64 to shift or sweep the center frequency (f₀) to make the cross-correlation coefficient r′ equal to or larger than the predetermined threshold.

First, as shown in step S71 in FIG. 22, the transmitters 61, 64 emit the radio waves having the narrowed bandwidth to the measurement target sites. Accordingly, as shown in step S72, the receivers 62, 63 receive the radio waves from the measurement target sites 91u, 91d. The processing proceeds to step S73, and the pulse wave detectors 101, 102 detect the pulse wave signals PS1, PS2.

Next, as shown in step S74 in FIG. 22, the third frequency controller 109 calculates in real time the cross-correlation coefficient r′ between the output waveform of the pulse wave signal PS1 output by the pulse wave detector 101 and the output waveform of the pulse wave signal PS2 output by the pulse wave detector 102. Furthermore, the third frequency controller 109 determines whether the calculated cross-correlation coefficient r′ exceeds the predetermined threshold Th2 (=0.99) (step S75 in FIG. 22). Here, if the cross-correlation coefficient r′ is equal to or smaller than the threshold Th2 (NO in step S75 in FIG. 22), the processing of steps S71 to S75 is repeated until the cross-correlation coefficient r′ exceeds the threshold Th2. When the cross-correlation coefficient r′ exceeds the threshold Th2 (YES in step S75 in FIG. 22), it is determined that the frequencies are appropriate, and the processing returns to the men-in flow (FIG. 9).

In this example, the similarity between the output waveform of the pulse wave detector 101 of the first set and the output waveform of the pulse wave detector 102 of the second set is increased, and thus the measurement accuracy of the pulse transit time (PTT) is improved.

In the above-described embodiments, the sphygmomanometer 1 is scheduled to be worn on the left wrist 90 as a measurement target site. However, the present disclosure is not limited to this. The measurement target site may be any portion through which an artery passes, and may be an upper limb such as a right wrist or an upper arm other than wrists, or a lower limb such as an ankle or a thigh.

In the above-described embodiments, the CPU 100 mounted on the sphygmomanometer 1 works as the pulse wave detectors and the first and second blood pressure calculators to execute the blood pressure measurement by the oscillometric method (the operation flow in FIG. 7B) and the blood pressure measurement (estimation) based on the PTT (the operation flow in FIG. 9). However, the present disclosure is not limited to this. For example, a substantial computer device such as a smartphone provided outside the sphygmomanometer 1 may work as the pulse wave detectors and the first and second blood pressure calculators to cause, via the network 900, the sphygmomanometer 1 to execute the blood pressure measurement by the oscillometric method (the operation flow in FIG. 7B) and the blood pressure measurement (estimation) based on the PTT (the operation flow in FIG. 9). In that case, the user can use an operation unit (touch panel, keyboard, mouse, etc.) of the computer device to perform an operation such as an instruction to start or stop the blood pressure measurement. The information on the blood pressure measurement such as a blood pressure measurement result or other information can be displayed on a display unit (organic EL display, LCD, etc.) of the computer device. In that case, in the sphygmomanometer 1, the display unit 50 and the operation unit 52 may be omitted.

Moreover, in an example of the present disclosure, an apparatus may include the pulse wave measurement device or the blood pressure measurement device, and further include a function unit that performs another function. According to this apparatus, it is possible to measure pulse wave with high accuracy, or calculate (estimate) a blood pressure value with high accuracy. In addition, this apparatus can perform various functions.

As described above, in the exemplary pulse wave measurement device of the present disclosure, a pulse wave measurement device configured to measure a pulse wave of a measurement target site of a living body, the pulse wave measurement device includes:

a transmitter configured to emit a radio wave toward the measurement target site;

a receiver configured to receive the radio wave reflected from the measurement target site; and

a pulse wave detector configured to detect, based on an output of the receiver, a pulse wave signal representing a pulse wave of an artery passing through the measurement target site and/or a tissue adjacent to the artery, wherein

the radio wave emitted from the transmitter has a bandwidth narrowed by a predetermined bandwidth index.

In the present specification, the “measurement target site” may be not only a rod-shaped portion such as an upper limb (wrist, upper arm, etc.) or a lower limb (ankle, etc.) but also a trunk.

The “tissue adjacent to an artery” refers to a portion of a living body that is adjacent to the artery and is periodically displaced under the influence of a pulse wave (that causes expansion and contraction of a blood vessel) of the artery.

The “bandwidth index” refers to, for example, an occupied bandwidth representing a range occupied by radio wave frequencies, a fractional bandwidth obtained by dividing the occupied bandwidth by a center frequency (f₀) (=occupied bandwidth/center frequency (f₀)), or the like. The bandwidth index is not limited to these, and another type of bandwidth index is possible.

When the “fractional bandwidth” is used as the “bandwidth index”, the fractional bandwidth is preferably 0.03 or smaller.

In the exemplary pulse wave measurement device of the present disclosure, the radio wave emitted from a transmitter has the bandwidth narrowed by the predetermined bandwidth index, and thus does not include wide frequency components as included in a square wave. Accordingly, the output of a receiver that receives the radio wave reflected from the measurement target site does not include wide frequency components as included in a square wave. Therefore, when the pulse wave detector detects, based on the output of the receiver, the pulse wave signal representing the pulse wave of the artery passing through the measurement target site and/or the tissue adjacent to the artery, it is possible to obtain the pulse wave signal having a high S/N ratio without requiring complicated signal processing such as the Fourier transform. That is, the pulse wave signal can be acquired with high accuracy.

Specifically, in the pulse wave measurement device based on a principle of capturing a phase change in the reflected wave due to a reflection position change resulting from a blood vessel diameter variation, using a radio wave having a wide bandwidth as in the prior art causes the phase change amount resulting from the blood vessel diameter variation to vary by frequency. The frequencies having these varied phase change amounts are superimposed and received, and thus signal processing such as the Fourier transform is required to detect the blood vessel diameter variation. Meanwhile, using a radio wave having a narrow bandwidth as in the present invention causes no superimposition of frequencies having the varied phase change amounts, and thus the phase change amount can be easily measured. Therefore, signal processing such as the Fourier transform is not required.

In the pulse wave measurement device of one embodiment, the transmitter intermittently transmits the radio wave having the narrowed bandwidth.

Since the pulse wave measurement device may be used for a portable electronic device, it is desirable that the power consumption is low. Thus, in the pulse wave measurement device according to this embodiment, the transmitter intermittently transmits the radio wave having the narrowed bandwidth. Accordingly, the receiver intermittently receives the radio wave reflected from the measurement target site. Therefore, the power consumption of the transmitter and the receiver is reduced and the power consumption of the pulse wave detector is also reduced as compared with the case of continuous transmission and reception.

In the pulse wave measurement device of one embodiment, the pulse wave measurement device comprises:

a first frequency controller configured to acquire a signal-to-noise ratio of the received signal, and perform control that causes the transmitter to shift or sweep a center frequency of the radio wave to make the acquired signal-to-noise ratio larger than a predetermined reference value.

Measurement environment of the pulse wave measurement device is under the influence of interference resulting from an individual difference of biological composition (personal difference in the case of a human body) or the like. For this reason, the measurement is sometimes difficult at a specific frequency. Thus, in the pulse wave measurement device according to this embodiment, the first frequency controller acquires the signal-to-noise ratio of the received signal, and performs the control that causes the transmitter to shift or sweep the frequency of the radio wave to make the acquired signal-to-noise ratio larger than the predetermined reference value. Accordingly, even if the measurement is difficult at a specific frequency due to an individual difference of biological composition, another frequency obtained by shifting or sweeping the frequency can be used. As a result, it is more likely that the pulse wave signal can be acquired with high accuracy.

In the pulse wave measurement device of one embodiment, the pulse wave measurement device comprises:

a second frequency controller configured to perform control that causes the transmitter to shift or sweep a center frequency (f₀) of the radio wave to make a cross-correlation coefficient between an output waveform of the pulse wave detector and a predetermined reference waveform equal to or larger than a predetermined threshold.

The “cross-correlation coefficient” means a sample correlation coefficient (also called Pearson's product-moment correlation coefficient). For example, when two sets of data string {xi} and data string (where i=1, 2, . . . , n) consisting of numerical values are given, the cross-correlation coefficient r between the data string {xi} and the data string {yi} is defined by an equation (Eq. 1) shown in FIG. 23. In the equation (Eq. 1), x and y with an upper bar represent average values of x and y, respectively.

In the pulse wave measurement device according to this embodiment, an output waveform when the pulse wave detector normally detects the pulse wave signal is set as the reference waveform in advance. Here, since the second frequency controller performs the control that causes the transmitter to shift or sweep the center frequency (f₀) of the radio wave to make the cross-correlation coefficient between the output waveform of the pulse wave detector and the reference waveform equal to or larger than the predetermined threshold, the similarity between the output waveform of the pulse wave detector and the reference waveform is increased. Therefore, the pulse wave signal can be acquired with high accuracy.

In the pulse wave measurement device of one embodiment, the pulse wave measurement device comprises:

a belt to be worn around the measurement target site, wherein the transmitter and the receiver are mounted on the belt to meet, in a wearing state where the belt is worn around an outer surface of the measurement target site, the artery passing through the measurement target site.

A user (including a subject; the same hereinafter) wears the pulse wave measurement device according to this embodiment on the measurement target site by winding the belt around the measurement target site. Thus, this pulse wave measurement device is stably worn on the measurement target site. In this wearing state, the transmitter emits the radio wave toward the artery of the measurement target site. The receiver receives the radio wave reflected from the artery of the measurement hand site and/or the tissue adjacent to the artery. The pulse wave detector detects, based on the output of the receiver, the pulse wave signal representing the pulse wave of the artery passing through the measurement target site and/or the tissue adjacent to the artery. Therefore, the pulse wave signal can be acquired with high accuracy.

In another aspect, the exemplary blood pressure measurement device of the present disclosure configured to measure blood pressure of a measurement target site of a living body, comprises:

two sets of the pulse wave measurement devices,

a belt of the two sets is integrally formed,

the transmitter and the receiver of a first set out of the two sets are disposed separately from the transmitter and the receiver of a second set in a width direction of the belt,

in a wearing state where the belt is worn around an outer surface of the measurement target site, the transmitter and the receiver of the first set meet an upstream portion of an artery passing through the measurement target site, while the transmitter and the receiver of the second set meet a downstream portion of the artery,

in each of the two sets, the transmitter emits a radio wave toward the measurement target site and the receiver receives the radio wave reflected from the measurement target site,

in each of the two sets, the pulse wave detector acquires, based on an output of the receiver, a pulse wave signal representing a pulse wave of the artery passing through the measurement target site and/or a tissue adjacent to the artery, and

the blood pressure measurement device comprises:

a time difference acquisition unit configured to acquire a time difference between the pulse wave signals acquired by the pulse wave detectors of the two sets as a pulse transit time; and

a first blood pressure calculator configured to calculate a blood pressure value based on the pulse transit time acquired by the time difference acquisition unit using a predetermined correspondence equation between pulse transit time and blood pressure.

In the exemplary blood pressure measurement device of the present disclosure, in the wearing state, the time difference acquisition unit can acquire the time difference between the pulse wave signals acquired by the pulse wave detectors of the two sets as the pulse transit time (PTT) with high accuracy. Therefore, the first blood pressure calculator can calculate (estimate) the blood pressure value with high accuracy.

In the blood pressure measurement device of one embodiment, each of the two sets includes a first frequency controller configured to acquire a signal-to-noise ratio of the received signal, and perform control that causes the transmitter to shift or sweep a center frequency of the radio wave to make the acquired signal-to-noise ratio larger than a predetermined reference value.

In the blood pressure measurement device according to this embodiment, in each of the two sets, even if the measurement is difficult at a specific frequency due to an individual difference of biological composition, another frequency obtained by shifting or sweeping the frequency can be used. As a result, it is more likely that the pulse wave signal can be detected with high accuracy.

In the blood pressure measurement device of one embodiment, each of the two sets includes a second frequency controller configured to perform control that causes the transmitter to shift or sweep a center frequency (f₀) of the radio wave to make a cross-correlation coefficient between an output waveform of the pulse wave detector and a predetermined reference waveform equal to or larger than a predetermined threshold.

In the blood pressure measurement device according to this embodiment, in each of the two sets, the similarity between the output waveform of the pulse wave detector and the reference waveform is increased, and thus the measurement accuracy of the pulse transit time (PTT) is improved.

In the blood pressure measurement device of one embodiment comprises:

a third frequency controller configured to perform control that causes the transmitter of the first set and/or the transmitter of the second set to shift or sweep a center frequency (f₀) of the radio wave to make a cross-correlation coefficient between an output waveform of the pulse wave detector of the first set and an output waveform of the pulse wave detector of the second set equal to or larger than a predetermined threshold.

In the blood pressure measurement device according to this embodiment, the similarity between the output waveform of the pulse wave detector of the first set and the output waveform of the pulse wave detector of the second set is increased, and thus the measurement accuracy of the pulse transit time (PTT) is improved.

In the blood pressure measurement device of one embodiment, a fluid bag for compressing the measurement target site is mounted on the belt, and

the blood pressure measurement device comprises:

a pressure controller configured to control pressure by supplying air into the fluid bag; and

a second blood pressure calculator configured to calculate blood pressure by an oscillometric method based on the pressure in the fluid bag.

In the blood pressure measurement device according to this embodiment, the blood pressure measurement (estimation) based on the pulse transit time (PTT) and the blood pressure measurement by the oscillometric method can be performed using the common belt. Therefore, user convenience is enhanced. Further, a rapid rise in blood pressure can be captured by the PTT method (blood pressure measurement based on the pulse transit time) that allows for continuous measurement with low accuracy. The rapid rise in blood pressure can be used as a trigger to start the measurement by the oscillometric method with higher accuracy.

In another aspect, the exemplary apparatus of the present disclosure comprises the pulse wave measurement device, or the blood pressure measurement device.

The exemplary apparatus of the present disclosure includes the pulse wave measurement device or the blood pressure measurement device, and may include a function unit that performs another function. According to this apparatus, it is possible to measure pulse wave with high accuracy, or calculate (estimate) a blood pressure value with high accuracy. In addition, this apparatus can perform various functions.

In another aspect, the exemplary pulse wave measurement method of the present disclosure for measuring a pulse wave of a measurement target site of a living body using the pulse wave measurement device comprises:

wearing the belt around an outer surface of the measurement target site to make the transmitter and the receiver meet an artery passing through the measurement target site;

emitting, by the transmitter, a radio wave having a bandwidth narrowed by a predetermined bandwidth index toward the measurement target site, and receiving, by the receiver, the radio wave reflected from the measurement target site; and

detecting, by the pulse wave detector, based on an output of the receiver, a pulse wave signal representing a pulse wave of the artery passing through the measurement target site and/or a tissue adjacent to the artery.

According to the exemplary pulse wave measurement method of the present disclosure, the radio wave emitted from the transmitter has the bandwidth narrowed by the predetermined bandwidth index, and thus does not include wide frequency components as included in a square wave. Accordingly, the output of the receiver that receives the radio wave reflected from the measurement hand site does not include wide frequency components as included in a square wave. Therefore, the pulse wave signal having a high signal-to-noise ratio (S/N ratio) can be obtained without requiring complicated signal processing such as the Fourier transform. That is, the pulse wave signal can be acquired with high accuracy.

In another aspect, the exemplary blood pressure measurement method of the present disclosure for measuring blood pressure of a measurement target site of a living body using the blood pressure measurement device comprises:

wearing the belt around an outer surface of the measurement target site to make the transmitter and the receiver of the first set out of the two sets meet an upstream portion of an artery passing through the measurement target site, and equally to make the transmitter and the receiver of the second set meet a downstream portion of the artery;

in each of the two sets, emitting, by the transmitter, a radio wave having a bandwidth narrowed by a predetermined bandwidth index toward the measurement target site, and receiving, by the receiver, the radio wave reflected from the measurement target site;

in each of the two sets, acquiring, by the pulse wave detector, based on an output of the receiver, a pulse wave signal representing a pulse wave of the artery passing through the measurement target site and/or a tissue adjacent to the artery;

acquiring, by the time difference acquisition unit, a time difference between the pulse wave signals acquired by the pulse wave detectors of the two sets as a pulse transit time; and

calculating, by the first blood pressure calculator, a blood pressure value based on the pulse transit time acquired by the time difference acquisition unit using a predetermined correspondence equation between pulse transit time and blood pressure.

According to this blood pressure measurement method, it is possible to acquire the pulse transit time (PTT) with high accuracy, and thus calculate (estimate) the blood pressure value with high accuracy.

The above embodiments are illustrative, and various modifications can be made without departing from the scope of the present invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A pulse wave measurement device configured to measure a pulse wave of a measurement target site of a living body, the pulse wave measurement device including:
a transmitter configured to emit a radio wave toward the measurement target site;
a receiver configured to receive the radio wave reflected from the measurement target site; and
a pulse wave detector configured to detect, based on an output of the receiver, a pulse wave signal representing a pulse wave of an artery passing through the measurement target site and/or a tissue adjacent to the artery, wherein
the radio wave emitted from the transmitter has a bandwidth narrowed by a predetermined bandwidth index.

2. The pulse wave measurement device according to claim 1, wherein
the transmitter intermittently transmits the radio wave having the narrowed bandwidth.

3. The pulse wave measurement device according to claim 1, comprising
a first frequency controller configured to acquire a signal-to-noise ratio of the received signal, and perform control that causes the transmitter to shift or sweep a center frequency of the radio wave to make the acquired signal-to-noise ratio larger than a predetermined reference value.

4. The pulse wave measurement device according to claim 1, comprising
a second frequency controller configured to perform control that causes the transmitter to shift or sweep a center frequency (f₀) of the radio wave to make a cross-correlation coefficient between an output waveform of the pulse wave detector and a predetermined reference waveform equal to or larger than a predetermined threshold.

5. The pulse wave measurement device according to claim 1, comprising
a belt to be worn around the measurement target site, wherein
the transmitter and the receiver are mounted on the belt to meet, in a wearing state where the belt is worn around an outer surface of the measurement target site, the artery passing through the measurement target site.

6. A blood pressure measurement device configured to measure blood pressure of a measurement target site of a living body, comprising
two sets of the pulse wave measurement devices according to claim 1, wherein
a belt of the two sets is integrally formed,
the transmitter and the receiver of a first set out of the two sets are disposed separately from the transmitter and the receiver of a second set in a width direction of the belt,
in a wearing state where the belt is worn around an outer surface of the measurement target site, the transmitter and the receiver of the first set meet an upstream portion of an artery passing through the measurement target site, while the transmitter and the receiver of the second set meet a downstream portion of the artery,
in each of the two sets, the transmitter emits a radio wave toward the measurement target site and the receiver receives the radio wave reflected from the measurement target site,
in each of the two sets, the pulse wave detector acquires, based on an output of the receiver, a pulse wave signal representing a pulse wave of the artery passing through the measurement target site and/or a tissue adjacent to the artery, and
the blood pressure measurement device comprises:
a time difference acquisition unit configured to acquire a time difference between the pulse wave signals acquired by the pulse wave detectors of the two sets as a pulse transit time; and
a first blood pressure calculator configured to calculate a blood pressure value based on the pulse transit time acquired by the time difference acquisition unit using a predetermined correspondence equation between pulse transit time and blood pressure.

7. The blood pressure measurement device according to claim 6, wherein
each of the two sets includes a first frequency controller configured to acquire a signal-to-noise ratio of the received signal, and perform control that causes the transmitter to shift or sweep a center frequency of the radio wave to make the acquired signal-to-noise ratio larger than a predetermined reference value.

8. The blood pressure measurement device according to claim 6, wherein
each of the two sets includes a second frequency controller configured to perform control that causes the transmitter to shift or sweep a center frequency (f₀) of the radio wave to make a cross-correlation coefficient between an output waveform of the pulse wave detector and a predetermined reference waveform equal to or larger than a predetermined threshold.

9. The blood pressure measurement device according to claim 6, comprising
a third frequency controller configured to perform control that causes the transmitter of the first set and/or the transmitter of the second set to shift or sweep a center frequency (f₀) of the radio wave to make a cross-correlation coefficient between an output waveform of the pulse wave detector of the first set and an output waveform of the pulse wave detector of the second set equal to or larger than a predetermined threshold.

10. The blood pressure measurement device according to claim 6, wherein
a fluid bag for compressing the measurement target site is mounted on the belt, and
the blood pressure measurement device comprises:
a pressure controller configured to control pressure by supplying air into the fluid bag; and
a second blood pressure calculator configured to calculate blood pressure by an oscillometric method based on the pressure in the fluid bag.

11. An apparatus comprising the pulse wave measurement device according to claim 1.

12. A pulse wave measurement method for measuring a pulse wave of a measurement target site of a living body using the pulse wave measurement device according to claim 5, comprising:
wearing the belt around an outer surface of the measurement target site to make the transmitter and the receiver meet an artery passing through the measurement target site;
emitting, by the transmitter, a radio wave having a bandwidth narrowed by a predetermined bandwidth index toward the measurement target site, and receiving, by the receiver, the radio wave reflected from the measurement target site; and
detecting, by the pulse wave detector, based on an output of the receiver, a pulse wave signal representing a pulse wave of the artery passing through the measurement target site and/or a tissue adjacent to the artery.

13. A blood pressure measurement method for measuring blood pressure of a measurement target site of a living body using the blood pressure measurement device according to claim 6, comprising:
wearing the belt around an outer surface of the measurement target site to make the transmitter and the receiver of the first set out of the two sets meet an upstream portion of an artery passing through the measurement target site, and equally to make the transmitter and the receiver of the second set meet a downstream portion of the artery;
in each of the two sets, emitting, by the transmitter, a radio wave having a bandwidth narrowed by a predetermined bandwidth index toward the measurement target site, and receiving, by the receiver, the radio wave reflected from the measurement target site;
in each of the two sets, acquiring, by the pulse wave detector, based on an output of the receiver, a pulse wave signal representing a pulse wave of the artery passing through the measurement target site and/or a tissue adjacent to the artery;
acquiring, by the time difference acquisition unit, a time difference between the pulse wave signals acquired by the pulse wave detectors of the two sets as a pulse transit time; and
calculating, by the first blood pressure calculator, a blood pressure value based on the pulse transit time acquired by the time difference acquisition unit using a predetermined correspondence equation between pulse transit time and blood pressure.
